(19) United States
(12) Patent Application Publication
(10) Pub. No.: US 2026/0265170 A1
(43) Pub. Date: Sep. 10, 2026

(54) METHODS AND INTERMEDIATES FOR PREPARING COMPOUNDS

(71) Applicant: VIIV HEALTHCARE UK (NO.5) LIMITED, Stevenage (GB)

(72) Inventors: Benedict James BARRON, Stevenage (GB); Sabine FENNER, Stevenage (GB); Stuart Grahame LEACH, Stevenage (GB); Matthew Allen ZAJAC, Collegeville, PA (US)

(73) Assignee: VIIV HEALTHCARE UK (NO.5) LIMITED, Stevenage (GB)

(21) Appl. No.: 19/491,407

(22) PCT Filed: Jun. 13, 2024
(86) PCT No.: PCT/IB2024/055798

§ 371 Date: Dec. 9, 2025

Related U.S. Application Data
(60) Provisional application No. 63/508,278, filed on Jun. 15, 2023.

(51) Int. Cl. C07C 45/72; C07C 49/427; C07C 49/477

(52) U.S. Cl. CPC C07C 45/72; C07C 49/427; C07C 49/477; C07B 2200/07; C07C 2602/18

(57) ABSTRACT

The disclosure relates to a process for the preparation of intermediates useful for preparing compounds that inhibit functions of the human immunodeficiency virus (HIV) during the virus replication cycle, such as disrupting functions of the capsid shell of HIV.

### FIELD OF THE INVENTION

The present invention relates to a process for the preparation of intermediates useful for preparing compounds that inhibit functions of the human immunodeficiency virus (HIV) during the virus replication cycle, such as disrupting functions of the capsid shell of HIV.

### BACKGROUND TO THE INVENTION

Acquired immunodeficiency syndrome (AIDS) is the result of HIV infection. HIV and AIDS continue to be major global public health issues. In 2021, an estimated 38.4 million people were living with HIV (including 1.7 million children)—a global HIV prevalence of 0.8%. The vast majority of this number live in low- and middle-income countries. In the same year, 650,000 people died of AIDS-related illnesses.

Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (Pls), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Despite the armamentarium of agents and drug combinations, there remains a medical need for new anti-retroviral agents. High viral heterogeneity, drug-associated toxicity, tolerability problems, and poor adherence can all lead to treatment failure and may result in the selection of viruses with mutations that confer resistance to one or more antiretroviral agents or even multiple drugs from an entire class (Beyrer, C., Pozniak A. “HIV drug resistance—an emerging threat to epidemic control.” N. Engl. J. Med. 2017, 377, 1605-1607; Gupta, R. K., Gregson J., et al. “HIV-1 drug resistance before initiation or re-initiation of first-line antiretroviral therapy in low-income and middle-income countries: a systematic review and meta-regression analysis.” Lancet Infect. Dis. 2017, 18, 346-355; Zazzi, M., Hu, H., Prosperi, M. “The global burden of HIV-1 drug resistance in the past 20 years.” PeerJ. 2018, DOI 10.7717/peerj.4848). As a result, new drugs are needed that are easier to take, have high genetic barriers to the development of resistance, and have improved safety over current agents. In this panoply of choices, novel mechanisms of action (MOAs) that can be used as part of the preferred antiretroviral therapy (ART) can still have a major role to play since they should be effective against viruses resistant to current agents. The improvements that would make drugs easier to take for long periods of time or even for a lifetime could include all or some of the following: reduced side effects, reduced drug-drug interactions, increased duration between dosing, or alternate routes of administration which match to individual patient preferences. The goals of improved safety would include high therapeutic indices towards any toxicities that would cause discontinuation of dosing, and could also include reduced side-effects or reduced drug-drug interactions. The potential to use fewer overall drugs in a combination regimen would also likely lead to improved compliance and safety. Increased potency against the antiviral target, especially if maintained in the presence of human plasma and serum albumin, would also lead to a reduced dose and could directly and positively affect the duration of dosing and the therapeutic index over side effects and toxicities. To summarize, maximum benefits to HIV-infected patients would be achieved if anti-HIV drugs with new mechanisms of action were discovered which also have the other benefits described above which facilitate long term compliance and safety.

Certain potentially therapeutic compounds which appear to act by disrupting the normal functions of the HIV capsid have been described in the art. Compounds acting through this mechanism would be useful additions to the options available for the treatment of HIV infection. Compounds which appear to target the HIV capsid have been the subject of recent reviews which describe much of the most important work to date. These reviews include the following: “HIV-1 Capsid Inhibitors as Antiretroviral Agents” Thenin-Houssier, Suzie; Valente, Susana T. Current HIV Research, 2016, 14, 270; “Inhibitors of the HIV-1 capsid, a target of opportunity” Carnes, Stephanie K.; Sheehan, Jonathan H.; Aiken, Christopher, Current Opinion in HIV & AIDS 2018, 13, 359-365; “HIV Capsid Inhibitors Beyond PF74” McArthur, Carole, Diseases, 2019, 7, 22; and “Insights into HIV-1 capsid inhibitors in preclinical and early clinical development as antiretroviral agents” Cevik, Muge; Orkin, Chloe Expert Opin Inv. Drugs, 2019, 28, 1021; and PCT patent applications with the following publication numbers: WO2012065062, WO2013006738, WO 2013006792, WO2014110296, WO2014110297, WO2014110298, WO2014134566, WO2015061518, WO2015130964, WO2015130966, WO2016040084, WO2016033243, WO2016172424, WO2016172425, WO2018035359, WO2018203235, WO2019035904, WO2019035973, WO2019161017, WO2019161280, WO2019198024, WO2020018459, WO2020053811, WO2020058844, WO2020084480, WO2020084491, WO2020084492, WO2020089778, WO2020095176, WO2020095177, WO2020157692, WO2020222108, WO2020254985, WO2021064570, WO2021064571, WO2021064677, WO2021070054, WO 2021176366, and WO2021176367.

What is now needed in the art are additional compounds which are novel and/or useful in the treatment of HIV. There is also a need to develop such compounds into an active pharmaceutical ingredient (API) that is suitable for use in drug manufacturing. Compound properties, such as solubility, hygroscopicity, and chemical/physical stability, are non-limiting factors used in drug development to obtain safe and effective drugs. Medicinal chemistry routes for the synthesis of new compounds often focus on diversity to access different analogues fast in small scale. In contrast, process chemistry routes for the manufacture of an API on industrial scale necessitate that factors such as scalability, overall yield, safety, environmental hazards, economy, and overall feasibility of the route be considered.

Thus, in another aspect, the present invention solves the problem of providing an efficient process (e.g., improved enantiomeric excess) for the manufacture of intermediate compounds which can be used, for example, in chemistry routes for producing HIV capsid inhibitors.

### SUMMARY OF THE INVENTION

The present invention relates to a process for the preparation of intermediates useful for preparing compounds that inhibit functions of HIV during the virus replication cycle, such as disrupting functions of the capsid shell of HIV.

In one aspect, the invention relates to a process for preparing a mixture comprising a compound of formula (I-a)

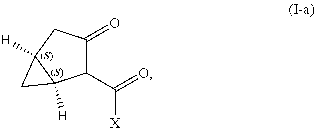

and a compound of formula (I-b)

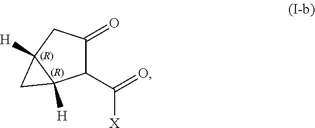

wherein the process comprises combining:
(a) a compound of formula (II),

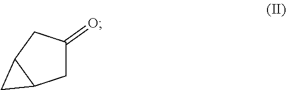

(b) a compound of formula (III),

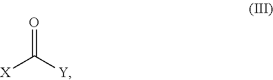

wherein X is cyclopropyl, difluoromethyl, or trifluoromethyl, and Y is a leaving group; and
   (c) a chiral amine;
   to provide the mixture.

In another aspect, the invention relates to a compound made by the above process.

### DETAILED DESCRIPTION OF THE INVENTION

### Definitions

The term “alkyl” refers to a saturated hydrocarbon radical, straight or branched, having the specified number of carbon atoms. For example, the term “C₁₋₆ alkyl” or “C₁-C₆ alkyl” refers to an alkyl group having 1 to 6 carbon atoms. Exemplary groups include, but are not limited to, methyl, ethyl, propyl (n-propyl and isopropyl), butyl (n-butyl, sec-butyl, isobutyl and tert-butyl), pentyl, and hexyl.

When the term “alkyl” is used in combination with other substituent groups, such as “halo(C₁₋₄)alkyl” and “hydroxy (C₁₋₄)alkyl,” the term “alkyl” is intended to encompass a divalent straight or branched chain hydrocarbon radical, wherein the point of attachment is through the alkyl moiety.

The term “cycloalkyl” refers to a non-aromatic, saturated, monocyclic, hydrocarbon ring containing the specified number of carbon atoms. For example, “cycloalkyl” may contain 3 to 8 carbon atoms, i.e., C₃₋₈ cycloalkyl. Exemplary groups include cyclopropyl, cyclobutyl, cyclopentyl, cyclohexyl, cycloheptyl or cyclooctyl.

The terms “halogen” and “halo” represent chloro, fluoro, bromo, or iodo substituents.

The term “cyano” refers to the group —CN.

The term “chiral amine” refers to an amine covalently bonded to a carbon atom, said carbon atom having three different substituents such that it is chiral. The term “chiral amine” is used interchangeably to describe the free base or ammonium salt form of the chiral amine. The chiral amine ammonium salt form refers to the ammonium species formed by the combination of the chiral amine free base with an acid.

The term “alkoxy” refers to an —O-alkyl group, i.e., an alkyl group which is attached through an oxygen linking atom, wherein “alkyl” is defined above. For example, the term “C₁₋₆ alkoxy” refers to an alkoxy group having 1 to 6 carbon atoms. Exemplary groups include, but are not limited to, methoxy, ethoxy, n-propoxy, isopropoxy, n-butoxy, s-butoxy, isobutoxy, and t-butoxy.

The term “cycloalkoxy” means a cycloalkyl group having the recited number of carbon atoms, with the same meaning as previously defined, attached via a ring carbon atom to an exocyclic oxygen atom. For example, “cycloalkoxy” may contain 3 to 6 carbon atoms attached via ring carbon atom to an exocyclic oxygen atom, i.e., C₃₋₆ cycloalkoxy. Exemplary groups include, but are not limited to, cyclopropoxyl, cyclobutoxyl, cyclopentoxyl, and cyclohexoxyl.

The term “allyl” refers to a moiety having the structural formula H₂C═CH—CH₂—*, where * denotes the point of attachment of the moiety to the remainder of the molecule, and the point of attachment is to a heteroatom or an aromatic moiety.

The term “aryl” refers to a monocyclic or bicyclic, hydrocarbon, aromatic radical. Aryl includes, for example, phenyl and naphthyl. An aryl group may contain 6 to 14 carbon atoms.

The term “heteroaryl” refers to a group or moiety comprising an aromatic monovalent monocyclic or bicyclic radical, containing 5 to 10 ring atoms, including at least one heteroatom independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including at least one heteroatom independently selected from nitrogen, oxygen and sulfur. Exemplary groups include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 5-membered “heteroaryl” groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl. Examples of 6-membered “heteroaryl” groups include oxo-pyridyl, pyridinyl, pyridazinyl, pyrazinyl, and pyrimidinyl. Examples of 6,6-fused “heteroaryl” groups include quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 6,5-fused “heteroaryl” groups include benzofuranyl, benzothienyl, benzimidazolyl, benzthiazolyl, indolizinyl, indolyl, isoindolyl, and indazolyl.

The term “chemical purity” means the overall level of the desired product or compound in the composition produced by the preparation. Where the compound is present in enantiomeric form, “chemical purity,” as used herein, includes both enantiomeric forms in the calculation of the overall level of the desired product. Components of the composition other than the desired product or compound are “impurities.” Purity may be measured by various techniques, including, but not limited to, HPLC analysis.

The term “enantiomeric purity” or “chiral purity” means the overall level of one enantiomer in a composition compared to the other enantiomer in the composition. Components of the composition other than any enantiomer are not considered in the calculation of “enantiomeric purity” or “chiral purity.” Enantiomeric or chiral purity may be measured by various techniques including, but not limited to, chiral SFC analysis and/or chiral HPLC analysis.

The term “enantiomeric excess” or “e.e.” refers to the percentage composition by which one enantiomer exceeds that of the other in a mixture of the two. For example, in a mixture containing 60% enantiomer A and 40% enantiomer B, the enantiomeric excess of enantiomer A is 20% (60% enantiomer A-40% enantiomer B=20% e.e.).

The term “optionally substituted” indicates that a group may be unsubstituted or substituted with one or more substituents as defined herein. The term “substituted” in reference to a group indicates that one or more hydrogen atoms attached to a member atom within a group is independently replaced by one or more of the defined substituents. In the case where groups may be selected from a number of alternative groups, the selected groups may be the same or different.

The term “independently selected” means that where more than one substituent is selected from a number of possible substituents, those substituents may be the same or different. Thus, each substituent is separately selected from the entire group of recited possible substituents.

The term “leaving group” generally refers to a group readily displaceable by a nucleophile such as an amine, a thiolate, an alkoxide, or an enolate. Examples of leaving groups include, but are not limited to, halides, imidazoles and pyridinium species.

The term “member atoms” refers to the atom or atoms that form a chain or ring. Where more than one member atom is present in a chain and within a ring, each member atom is covalently bound to an adjacent member atom in the chain or ring. Atoms that make up a substituent group attached to a chain or ring are not member atoms in the chain or ring.

The term “aprotic solvent” means a solvent molecule that neither accepts nor provides a proton.

### EMBODIMENTS OF THE INVENTION

In one aspect, the invention relates to a process for preparing a mixture comprising a compound of formula (I-a)

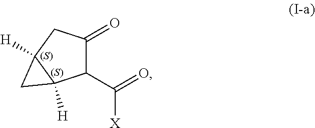

and a compound of formula (I-b)

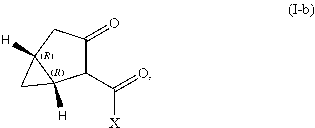

wherein the process comprises combining:
(a) a compound of formula (II),

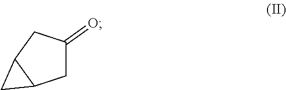

(b) a compound of formula (III),

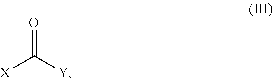

wherein X is cyclopropyl, difluoromethyl, or trifluoromethyl, and Y is a leaving group; and
   (c) a chiral amine;
   to provide the mixture.

In embodiment, the invention relates to a process for preparing a mixture comprising a compound of formula (I-a)

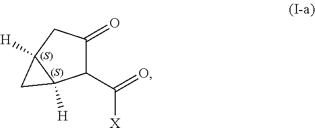

and a compound of formula (I-b)

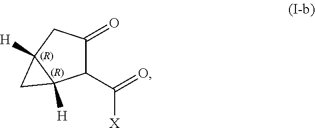

wherein the process comprises combining:
(a) a compound of formula (II),

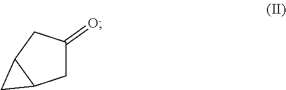

(b) a compound of formula (III),

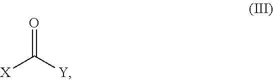

wherein X is cyclopropyl, difluoromethyl, or trifluoromethyl, and Y is a leaving group;
   (c) a chiral amine;
   (d) a lithium base;
   (e) optionally, a lithium salt; and
   (f) an aprotic solvent;
   to provide the mixture.

In an aspect of the invention the compound of formula (III) is

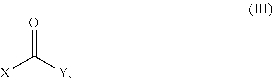

wherein X is cyclopropyl, difluoromethyl, or trifluoromethyl, and Y is a leaving group. As earlier described, the term “leaving group” generally refers to a group readily displaceable by a nucleophile such as an amine, a thiolate, an alkoxide, or an enolate. Such leaving groups are well known in the art and one skilled in the art will appreciate that many possible leaving groups may be used. Examples of such leaving groups include, but are not limited to, halides, imidazoles and pyridinium species. Leaving groups can also be described in reference to the chemical that is released when Y of the compound of formula (III) is displaced by a nucleophile. For example, following displacement by a nucleophile, Y can take the form of an alcohol, phenol, carboxylic acid, N-hydroxysuccinimide, N-hydroxybenzotriazole, or the conjugate base thereof.

In an embodiment of the invention the compound of formula (III) is the product of a carboxylic acid reacting with an “activating agent” according to the formula below:

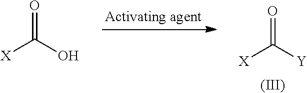

wherein X is cyclopropyl, difluoromethyl, or trifluoromethyl and the “activating agent” is one of many reagents known in the art capable of generating a leaving group (Y) in this transformation. One skilled in the art will appreciate that many activating agents may be used. Examples of such activating agents include, but are not limited to, 1-[3-(dimethylamino) propyl]-3-ethylcarbodiimide hydrochloride (EDC), O-(7-azabenzotriazol-1-yl)-1,1,3,3-tetramethyluronium hexafluorophosphate (HATU), 2-propanephosphonic acid anhydride (T3P), 1-[(1-(cyano-2-ethoxy-2-oxoethylideneaminooxy)-dimethylamino-morpholinomethylene)]methanaminium hexafluorophosphate (COMU), and benzotriazol-1-yloxytri (pyrrolidino) phosphonium hexafluorophosphate (PyBOP). Some possible activating agents are also described in Chem. Rev. 2011, 111 (11), 6557-6602.

In another embodiment of the invention, Y is selected from —F, —Cl, —Br, —I, —CN, C₁-C₆ alkoxy, C₃-C₆ cycloalkoxy, —O(CO)C₁-C₆ alkyl, —O(CO)C₃-C₆ cycloalkyl, —O-aryl, or —O-heteroaryl; wherein —O(CO) C₁-C₆ alkyl or —O(CO)C₃-C₆ cycloalkyl may optionally be substituted by 1, 2, or 3 fluorine atoms, and wherein O-aryl and O-heteroaryl may be optionally substituted 1, 2, or 3 times by substituents independently selected from halogen atoms or C₁-C₃ alkyl.

In an embodiment of the invention, Y is chloro, methoxy, or ethoxy. In another embodiment of the invention, Y is methoxy or ethoxy.

In an aspect of the invention, a combination of the chiral amine, the lithium base, and, optionally, the lithium salt, in the presence of the solvent forms a lithium amide base.

In an embodiment, the lithium base is selected from Li—C₁-C₆-alkyl, Li—C₅-C₆-cycloalkyl, Li (aryl), LiH, LiNH₂, or lithium metal (Li⁰). In another embodiment, the lithium base is selected from n-Butyllithium (n-BuLi), sec-Butyllithium (sec-BuLi), isopropyllithium (i-PrLi) or tert-Butyllithium (t-BuLi). In another embodiment, the lithium base is n-BuLi.

In an embodiment, the solvent is any solvent that may coordinate, but does not undergo a chemical reaction, with any of the components with which it is combined. One skilled in the art will appreciate that many solvents may be used alone or in combination with one another. In an embodiment, the solvent is an aprotic or non-protogenic solvent. Non-limiting examples of such solvents include tetrahydrofuran (THF), 1,4-dioxane, 2-methyltetrahydrofuran, diethyl ether, methyl tert-butyl ether (MTBE), and cyclopentyl methyl ether (CPME). In an embodiment, the solvent is selected from THF, 1,4-dioxane, 2-methyltetrahydrofuran, diethyl ether, MTBE, or CPME. In another embodiment, the solvent is selected from THF, 1,4-dioxane, 2-methyltetrahydrofuran, MTBE, or CPME. In another embodiment, the solvent is THF.

In an embodiment, the lithium salt is present. In another embodiment, the lithium salt is LiCl or LiBr. In an embodiment, there is no lithium salt.

As previously described, the term “chiral amine” refers to an amine covalently bonded to a carbon atom, said carbon atom having three different substituents such that it is chiral. The term “chiral amine” is used interchangeably to describe the free base or ammonium salt form of the chiral amine. The chiral amine ammonium salt form refers to the ammonium species formed by the combination of the chiral amine free base with an acid.

In an aspect of the invention, a combination of a chiral amine, a lithium base, and optionally, a lithium salt, in the presence of a solvent forms a lithium amide base. In an embodiment, the lithium salt is present. In another embodiment, a molar ratio of the chiral amine and the lithium salt is in a range of from about 4:1 to about 2:5. In another embodiment, the molar ratio of the chiral amine and the lithium salt is in a range of from about 6:5 to about 1:1.

In an embodiment, a combination of a chiral amine free base, a lithium base, and, optionally, a lithium salt, in the presence of a solvent forms a lithium amide base. In another embodiment, a molar ratio of the chiral amine free base and lithium base is in a range from about 9:10 to about 5:6. In another embodiment, the molar ratio of the chiral amine free base and the lithium base is in a range from about 1.0:1.0 to about 1.0:1.1.

In another embodiment, a combination of a chiral amine ammonium salt, a lithium base, and, optionally, a lithium salt, in the presence of a solvent forms a lithium amide base. In an embodiment, a molar ratio of the chiral amine ammonium salt and lithium base is in a range from about 1.0:1.8 to about 1.0:1.0:2.4. In another embodiment, the molar ratio of the chiral amine ammonium salt and lithium base is in a range from about 1.0:2.0 to about 1.0:2.2.

In one embodiment of the invention, the lithium base is n-BuLi, the lithium salt is present and is LiCl, and the solvent is THF.

In one embodiment of the invention, the chiral amine is a compound selected from formula (IV-a), (IV-b), (IV-c), (IV-d), (IV-e), or (IV-f):

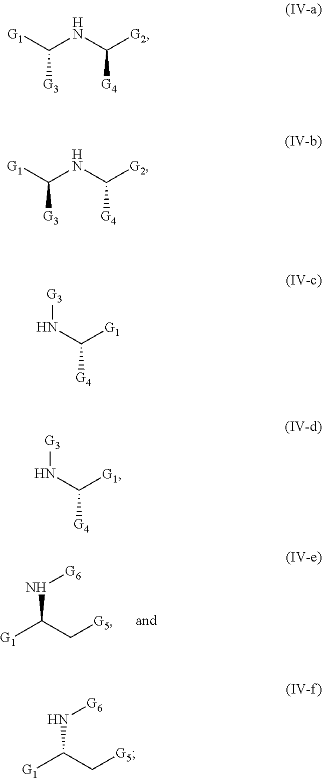

wherein
   G₁ is unsubstituted phenyl or phenyl substituted by one, two, or three substituents independently selected from halogen, cyano, substituted C₁-C₄ alkyl, unsubstituted C₁-C₄ alkyl, —O (cyclopropyl), —O (allyl), allyl, —O(R₁), or —C(O)(R₁);
   G₂ is unsubstituted phenyl or phenyl substituted by one, two, or three substituents independently selected from halogen, cyano, substituted C₁-C₄ alkyl, unsubstituted C₁-C₄ alkyl, —O (cyclopropyl), —O (allyl), allyl, —O(R₁), or —C(O) (R₁);
   G₃ is unsubstituted C₁-C₃ alkyl or C₁-C₃ alkyl substituted by one, two, or three halogen atoms;
   G₄ is unsubstituted C₁-C₃ alkyl or C₁-C₃ alkyl substituted by one, two, or three halogen atoms;
   G₅ is selected from azetidine, pyrrolidine, morpholine, piperidine, or quinuclidine, each of which is optionally substituted by one or two substituents independently selected from methyl or halogen;
   G₆ is selected allyl; cyclopropyl; unsubstituted C₁-C₅ alkyl; or C₁-C₅ alkyl substituted either by one phenyl or by one, two, or three halogen atoms;
   R₁ is unsubstituted C₁-C₄ alkyl or C₁-C₄ alkyl substituted by one, two, or three halogen atoms.

In one embodiment of the invention, the chiral amine is (R)-bis((R)-1-phenylethyl)amine,

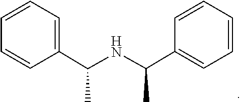 [SMILES: C[C@@H](N[C@H](C)c1ccccc1)c1ccccc1]

or (R)-bis((R)-1-phenylethyl)amine hydrochloride,

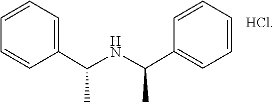 [SMILES: C[C@@H](N[C@H](C)c1ccccc1)c1ccccc1]

In one embodiment of the invention a lithium amide base formed by the combination of a chiral amine, lithium base and optional lithium salt in a solvent (“Solvent A”) is next combined with bicyclo[3.1.0]hexan-3-one in a solvent (“Solvent B”). “Solvent A” and “Solvent B” are any solvent which does not undergo a chemical reaction with any of the components with which it is combined. One skilled in the art will appreciate that many solvents may be used, and that “Solvent A” and “Solvent B” may be the same or different. Examples of such solvents include, but are not limited to, THF, 1,4-dioxane, 2-methyltetrahydrofuran, diethyl ether, MTBE, and CPME. One skilled in the art will recognize that the solvent(s) used and the environment in which the solvent(s) is used must be largely free of water prior to quenching and workup of the reaction mixture. In an embodiment, the solvent(s) used prior to quenching and workup of the reaction mixture contains below 500 ppm of water. In another embodiment, the solvent(s) used prior to quenching and workup of the reaction mixture contains below 300 ppm water. In another embodiment, the solvent(s) used prior to quenching and workup of the reaction mixture contains below 150 ppm water.

In an embodiment of the invention, the compound of formula (I-a) in the mixture produced by the process described herein is present in an enantiomeric excess over the compound of formula (I-b) of equal to or greater than 5%, equal to or greater than 10%, equal to or greater than 15%, equal to or greater than 20%, equal to or greater than 25%, equal to or greater than 30%, equal to or greater than 35%, equal to or greater than 40%, equal to or greater than 45%, equal to or greater than 50%, equal to or greater than 55%, equal to or greater than 60%, equal to or greater than 65%, equal to or greater than 70%, equal to or greater than 75%, equal to or greater than 80%, or equal to or greater than 85%.

In one embodiment of the invention, the differential solubility of two compounds present in a mixture is used to selectively enrich one compound over the other. Upon treatment of the mixture with a solvent such that some solid material is dissolved and some solid material remains, the ratio of the two components in the solid phase versus liquid phases becomes different. This process is known as “trituration.”

In another embodiment, when two compounds are present in a mixture, trituration may be used to selectively enrich the amount of one compound versus the other in the solid phase, and the solids are then isolated by filtration.

In a further embodiment, when two regioisomers are present in a mixture, trituration may be used to selectively enrich the amount of one regioisomer versus the other in the solid phase, and the solids are then isolated by filtration.

In another embodiment, when two regioisomers present in a mixture contain a carboxylic acid group, the carboxylic acid group is converted to a corresponding lithium acetate group before trituration.

In a further embodiment, when two lithium acetate regioisomers are present in a mixture, trituration may be used to selectively enrich the amount of one regioisomer versus the other in the solid phase, and the solids are then isolated by filtration.

In another embodiment, when trituration is used to enrich the amount of one compound versus the other in the solid phase and the solids are then isolated by filtration, the filtrate may also be separately isolated and further processed in a subsequent step to provide additional material.

In one embodiment of the invention, a mixture of the regioisomers lithium 2-((3bS,4aS)-3-cyclopropyl-3b,4,4a,5-tetrahydro-1H-cyclopropa[3,4]cyclopenta[1,2-c]pyrazol-1-yl)acetate and lithium 2-((3bS,4aS)-3-cyclopropyl-3b,4,4a,5-tetrahydro-2H-cyclopropa[3,4]cyclopenta[1,2-c]pyrazol-2-yl)acetate,

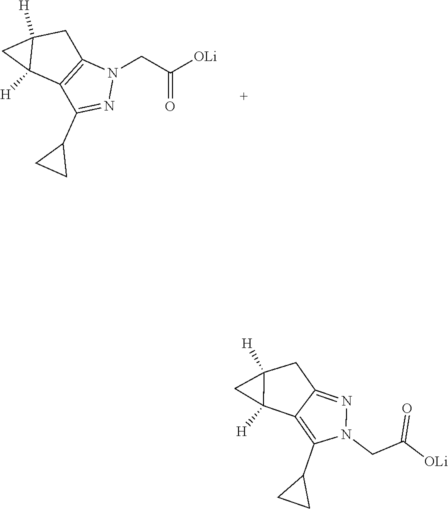

is subjected to a trituration process resulting in enrichment in the solid phase of one regioisomer over the other.

In another embodiment of the invention, a mixture of the regioisomers lithium 2-((3bS,4aS)-3-cyclopropyl-3b,4,4a,5-tetrahydro-1H-cyclopropa[3,4]cyclopenta[1,2-c]pyrazol-1-yl)acetate and lithium 2-((3bS,4aS)-3-cyclopropyl-3b,4,4a,5-tetrahydro-2H-cyclopropa[3,4]cyclopenta[1,2-c]pyrazol-2-yl)acetate,

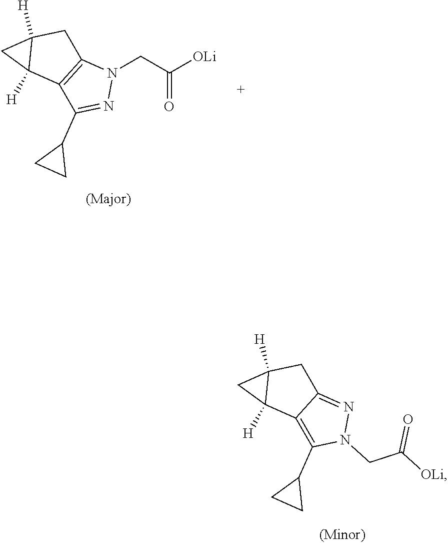

where lithium 2-((3bS,4aS)-3-cyclopropyl-3b,4,4a,5-tetrahydro-1H-cyclopropa[3,4]cyclopenta[1,2-c]pyrazol-1-yl)acetate is the major (>50% wt) regioisomer present, is subjected to a trituration process to afford lithium 2-((3bS,4aS)-3-cyclopropyl-3b,4,4a,5-tetrahydro-1H-cyclopropa[3,4]cyclopenta[1,2-c]pyrazol-1-yl)acetate,

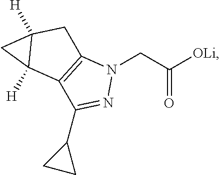

in >90% chemical purity.

In one embodiment of the invention, the trituration process used to separate two regioisomers uses water as one component of the solvent.

In another embodiment of the invention, the trituration process used to separate two regioisomers uses aqueous acid as one component of the solvent.

In another embodiment of the invention, the trituration process used to separate two regioisomers uses aqueous acid as the solvent.

In another embodiment of the invention, the trituration process used to separate two regioisomers uses aqueous HCl as the solvent.

In another embodiment of the invention, the trituration process used to separate two regioisomers uses aqueous 1N HCl as the solvent.

In another embodiment of the invention, a compound containing the lithium acetate group, when treated with aqueous acid, maintains the lithium acetate group.

In another embodiment of the invention, a solid compound containing the lithium acetate group, when treated with aqueous acid, maintains the lithium acetate group.

In another embodiment of the invention, a solid mixture of the regioisomers lithium 2-((3bS,4aS)-3-cyclopropyl-3b,4,4a,5-tetrahydro-1H-cyclopropa[3,4]cyclopenta[1,2-c]pyrazol-1-yl)acetate and lithium 2-((3bS,4aS)-3-cyclopropyl-3b,4,4a,5-tetrahydro-2H-cyclopropa[3,4]cyclopenta[1,2-c]pyrazol-2-yl)acetate, where

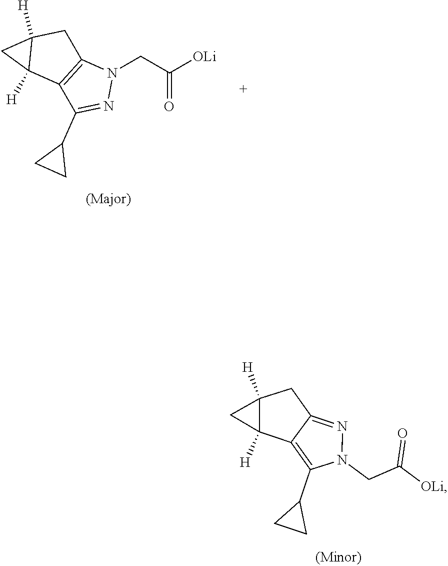

lithium 2-((3bS,4aS)-3-cyclopropyl-3b,4,4a,5-tetrahydro-1H-cyclopropa[3,4]cyclopenta[1,2-c]pyrazol-1-yl)acetate is the major (>50% wt) regioisomer present, is subjected to a trituration process using aqueous HCl to afford lithium 2-((3bS,4aS)-3-cyclopropyl-3b,4,4a,5-tetrahydro-1H-cyclopropa[3,4]cyclopenta[1,2-c]pyrazol-1-yl)acetate,

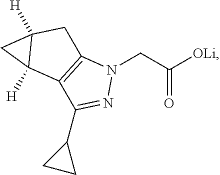

in >90% chemical purity.

### Additional Processes

As may be appreciated by one of skill in the art, compounds of formula (I-a) and (I-b) are intermediates, of which either or both may be useful in additional processes. For example, WO 2020/054492, which is directed to novel capsid inhibitors, describes, inter alia, preparation of intermediate 2-(2,2-difluoroacetyl) bicyclo[3.1.0]hexan-3-one,

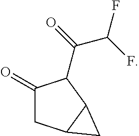 [SMILES: O=C1CC2CC2C1C(=O)C(F)F]

The process described herein may be used to prepare a mixture of enantiomers of 2-(2,2-difluoroacetyl) bicyclo[3.1.0]hexan-3-one, corresponding to instant compounds of formula (I-a) and (I-b), where X is difluoromethyl. Thus, the process described herein may be useful to the processes described in WO 2020/054492 by providing mixtures with desired enantiomeric excesses, thereby reducing resources required to produce desired intermediates.

Similarly, U.S. Pat. No. 10,696,657, which is directed to antiretroviral compounds and processes for making the same, describes, inter alia, processes of preparing 2-((3bS,4aR)-5,5-difluoro-3-(trifluoromethyl)-3b,4,4a,5-tetrahydro-1H-cyclopropa[3,4]cyclopenta[1,2-c]pyrazol-1-yl) acetic acid,

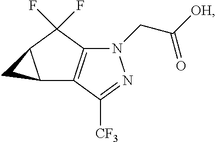 [SMILES: O=C(O)Cn1nc(C(F)(F)F)c2c1C(F)(F)[C@@H]1C[C@H]21]

which is an intermediate used in synthetic processes further described in U.S. Pat. No. 10,696,657. The process described herein may also be used, for example, to prepare a mixture of enantiomers of 2-(2,2,2-trifluoroacetyl) bicyclo[3.1.0]hexan-3-one,

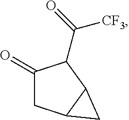 [SMILES: O=C1CC2CC2C1C(=O)C(F)(F)F]

corresponding to instant compounds of formula (I-a) and (I-b), where X is trifluoromethyl. Either or both enantiomers from this mixture may, in turn, be used in processes of preparing 2-((3bS,4aR)-5,5-difluoro-3-(trifluoromethyl)-3b,4,4a,5-tetrahydro-1H-cyclopropa[3,4]cyclopenta[1,2-c]pyrazol-1-yl) acetic acid. Thus, the process described herein may be useful to the processes described in U.S. Pat. No. 10,696,657 by providing mixtures with desired enantiomeric excesses, thereby reducing resources required to produce desired intermediates.

### General Synthetic Routes

One embodiment of the method of this invention can be summarized by the following reaction schemes, wherein W, X, Y, and Z are as defined above.

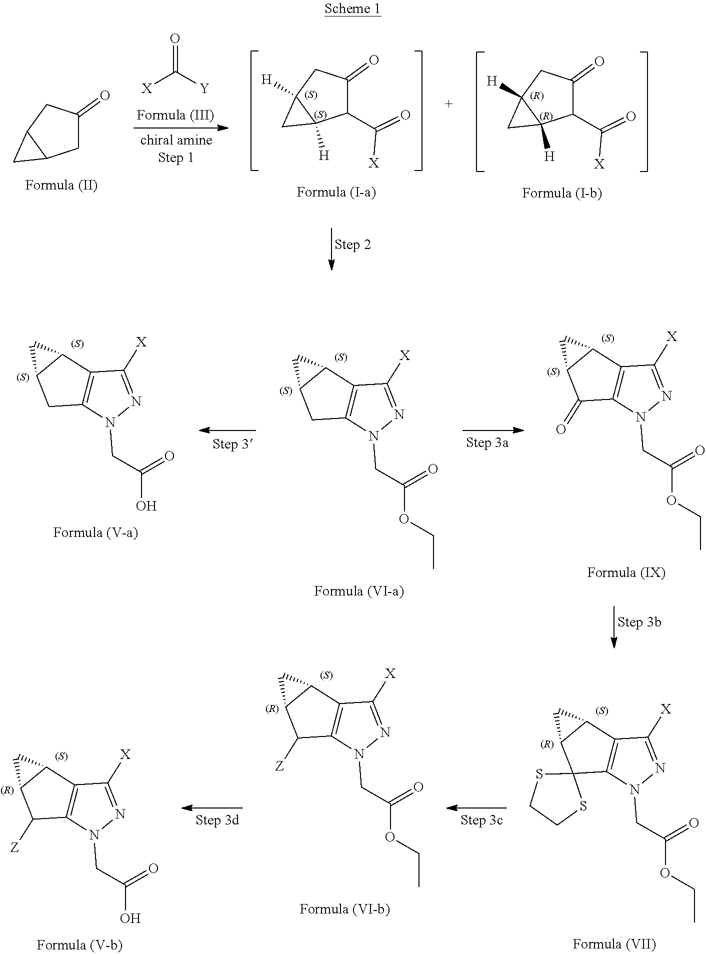

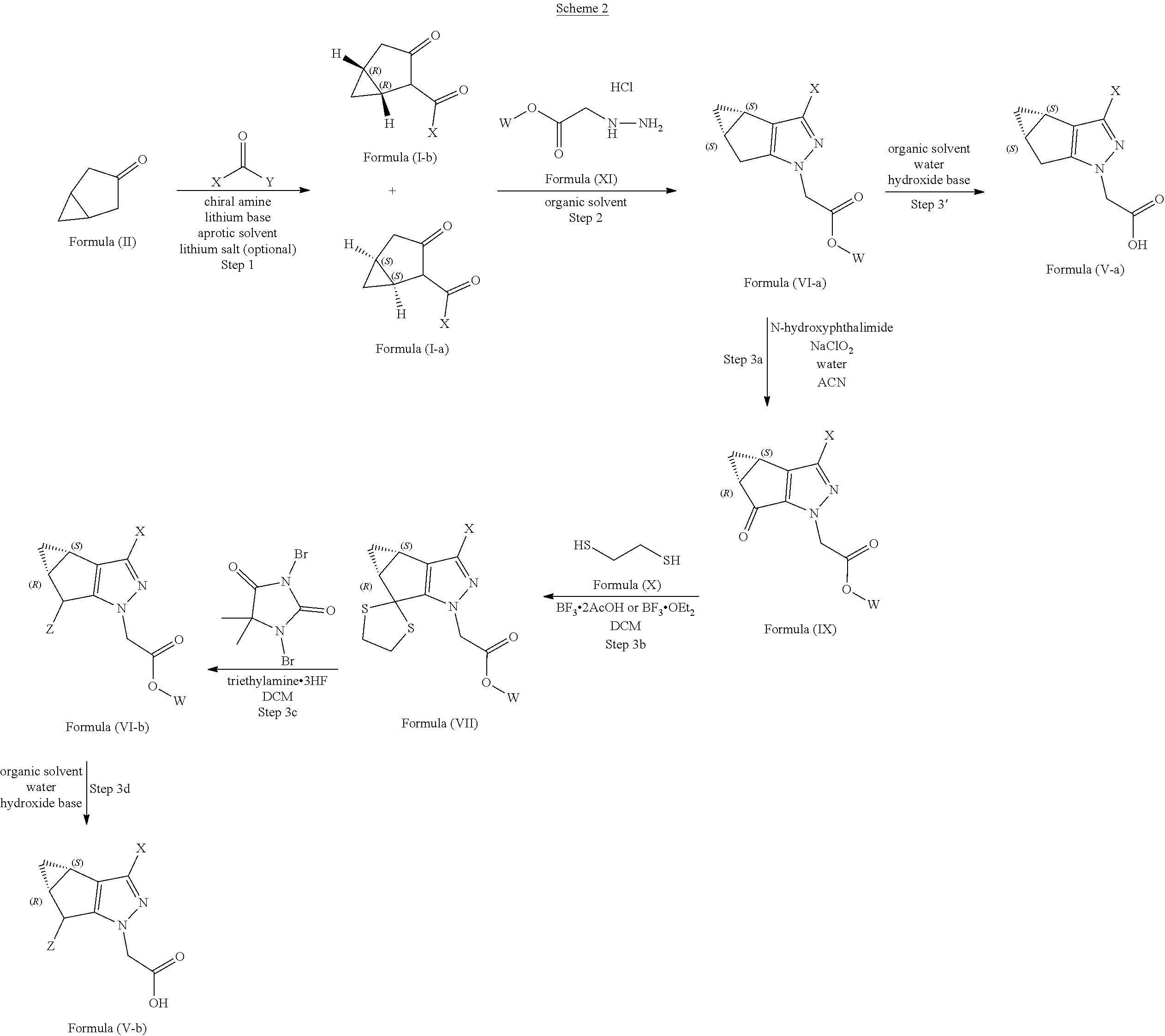

The method of this invention is further illustrated by the following examples. Examples 1, 2, and 3 are summarized in the three schemes below:

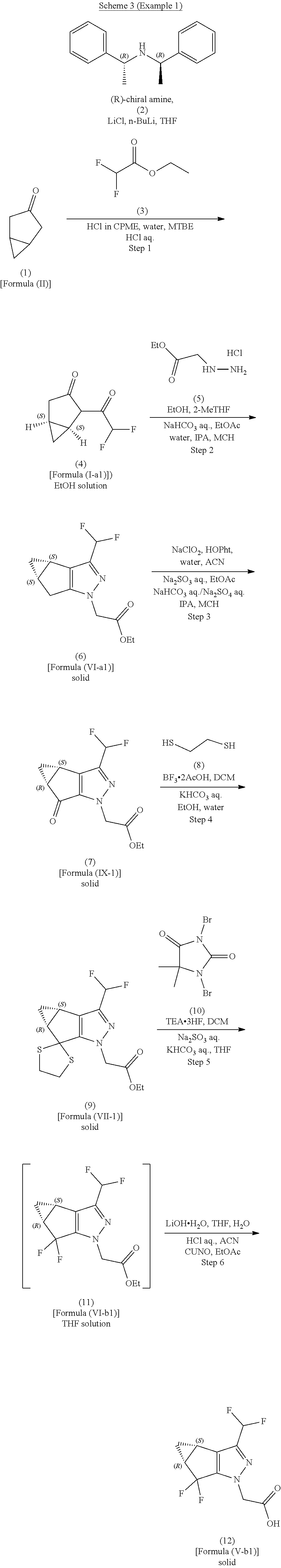

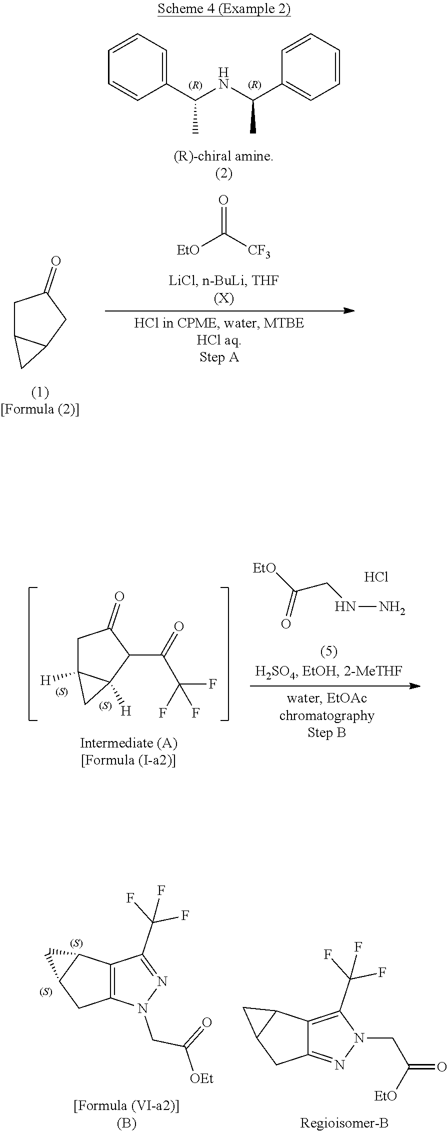

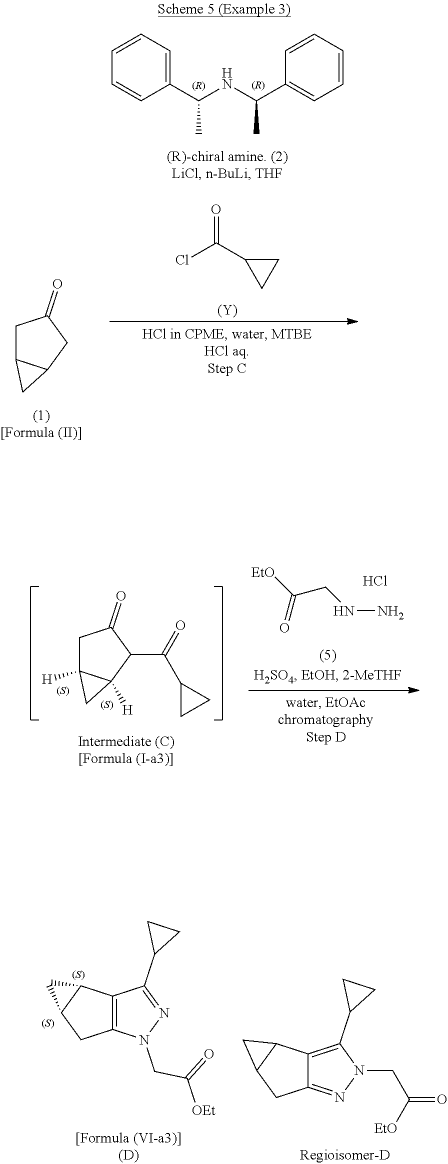

### EXAMPLES

### Abbreviations

ACN Acetonitrile
AcOH Acetic acid
CDCl₃ Chloroform
CPME cyclopentyl methyl ether
CUNO Filtration cartridge
DBDMH 1,3-Dibromo-5,5-Dimethylhydantoin
DCM Dichloromethane
EtOAc Ethyl acetate
EtOH Ethanol
HCl Hydrochloric acid
HOPht N-Hydroxyphthalimide
HPLC High-performance liquid chromatography
IPA Isopropanol
MCH Methylcyclohexane
2-MeTHF 2-Methyltetrahydrofuran
MTBE Methyl tert-butyl ether
SFC Super-critical fluid chromatography (using carbon dioxide)
TEA.3HF Triethylamine trihydrofluoride
TFA Trifluoroacetic acid
THF Tetrahydrofuran

### Example 1

Note: 1 wt is defined as the weight of bicyclo[3.1.0]hexan-3-one (1) (step 1) and of the respective previous step title compound (steps 2-6) to the reaction vessel in grams. All other weights, volumes and equivalents given are calculated relative to this figure.

### Step 1: Synthesis of Intermediate of Formula (I-a1) ((1S,5S)-2-(2,2-difluoroacetyl) bicyclo[3.1.0]hexan-3-one) (4)

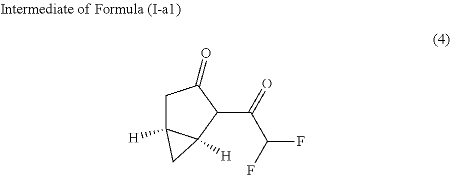 [SMILES: O=C1C[C@@H]2C[C@@H]2C1C(=O)C(F)F]

(R)-bis((R)-1-phenylethyl)amine hydrochloride (2) (1.2 eq.) was charged to a reaction vessel equipped with stirrer and internal thermometer. THF (6.3 vol) was added, followed by LiCl (1.0 eq.), then the reaction vessel was degassed and purged with nitrogen for 3 times. The vessel was cooled in an acetone/dry ice bath until internal reading reached-65~−75° C. n-BuLi (2.3 eq., 2.5M) was added dropwise whilst maintaining the internal temperature <−60° C. The contents were stirred at −65~−75° C. for 10 minutes then warmed to 20° C. over 1 hour. The contents were then cooled back to −65~−75° C. In a separate vessel a solution of bicyclo[3.1.0]hexan-3-one (1) (55 g, 1 wt., 1.0 eq.) in THF (1.3 vol) was prepared and the vessel was purged with nitrogen for 10-15 minute. The freshly prepared ketone solution was added dropwise to the lithium amide base solution at −65~−75° C., whilst maintaining the internal temperature <−65° C. The contents were stirred for 20 minutes at −65~−75° C. Next ethyl 2,2-difluoroacetate (3) (1.5 eq.) in THF (1.3 vol) was added dropwise at −65~−75° C., whilst maintaining the internal temperature <−65° C. The contents were stirred at −65~−75° C. for 1-2 hours. The reaction was quenched with HCl (15.9 vol, 3M solution in CPME) by fast addition to the cold mixture. The mixture was then allowed to warm to 0° C. whilst stirring. Water (12.5 vol) and MTBE (12.5 vol) were added and the solution was stirred for 10 minutes. The phases were allowed to separate, then the aqueous phase was discharged. The organic phase was washed with 2M HCl (3×8 vol), then water (2×8 vol). The organic phase was dried over Na₂SO₄, filtered and the solvent was evaporated in vacuo to obtain crude ((1S,5S)-2-(2,2-difluoroacetyl) bicyclo[3.1.0]hexan-3-one) (4) as an oil (93.5 g, 94.1% chemical purity and 85.4% chiral purity, 70.4% assay yield).

¹H NMR (400 MHz, CDCl₃-d) δ ppm 5.90-6.47 (m, 1H), 2.80 (dd, J=19.93, 6.89 Hz, 1H), 2.46 (d, J=19.93 Hz, 1H), 2.22-2.36 (m, 1H), 1.64-1.81 (m, 1H), 1.12-1.22 (m, 2H), 0.33 (q, J=4.35 Hz, 1H).

### Step 2: Synthesis of Intermediate of Formula (VI-a1) (ethyl 2-((3bS,4aS)-3-(difluoromethyl)-3b,4,4a,5-tetrahydro-1H-cyclopropa[3,4]cyclopenta[1,2-c]pyrazol-1-yl)acetate) (6)

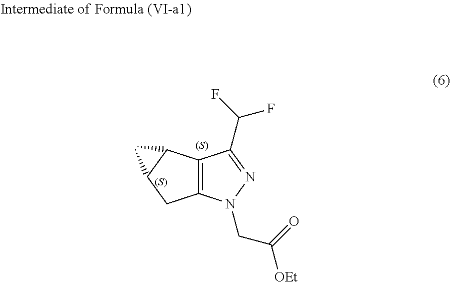

Aminoglycinate hydrochloride (5) (1.2 eq.) was charged to a reaction vessel. 2-MeTHF (15 vol) was added and the vessel was purged with nitrogen for 10-15 minutes. The contents were cooled to 10° C. In a separate vessel a solution of ((1S,5S)-2-(2,2-difluoroacetyl) bicyclo[3.1.0]hexan-3-one (4) (99.6 g, 1 wt., 1.0 eq.) in EtOH (15 vol) was prepared. The freshly prepared solution of ((1S,5S)-2-(2,2-difluoroacetyl) bicyclo[3.1.0]hexan-3-one (4) was added to the hydrazine containing reaction vessel at 10-15° C. The mixture was allowed to warm to 10-20° C. until complete consumption of starting material (by GC) was observed. The mixture was added into a 5% aqueous NaHCO₃ solution (15 wt.) at 0-10° C., then EtOAc (25 vol) was added. The phases were allowed to separate. The aqueous phase was discharged. The organic phase was washed with water (10 vol), then concentrated to about 8-10 vol, 2-MeTHF (10 vol) was charged, then the solvent was exchanged with IPA (2×3 vol) and concentrated to 2 vol. MCH (4 vol) was charged dropwise at 20° C., then the contents were cooled to 0° C. and stirred for 1 hour. The mixture was filtered, the cake was washed with MCH (1 vol) and dried at 35° C. in vacuo to obtain (ethyl2-((3bS,4aS)-3-(difluoromethyl)-3b,4,4a,5-tetrahydro-1H-cyclopropa[3,4]cyclopenta[1,2-c]pyrazol-1-yl)acetate) (6) as a pale yellow solid (57.2 g, 99.9% chemical purity and 99.5% chiral purity, 39.5% assay yield). 1H NMR (400 MHz, CDCl₃-d) δ ppm 6.38-6.84 (m, 1H), 4.72 (d, J=8.37 Hz, 2H), 4.24 (q, J=7.14 Hz, 2H), 2.83-3.00 (m, 1H), 2.64-2.79 (m, 1H), 2.06-2.24 (m, 2H), 1.30 (t, J=7.14 Hz, 3H), 1.12 (td, J=7.75, 4.92 Hz, 1H), 0.33 (q, J=4.35 Hz, 1H).

### Step 3: Synthesis of Intermediate of Formula (IX-1) (ethyl 2-(3-(difluoromethyl)-5-oxo-3b,4,4a,5-tetrahydro-1H-cyclopropa[3,4]cyclopenta[1,2-c]pyrazol-1-yl)acetate) (7)

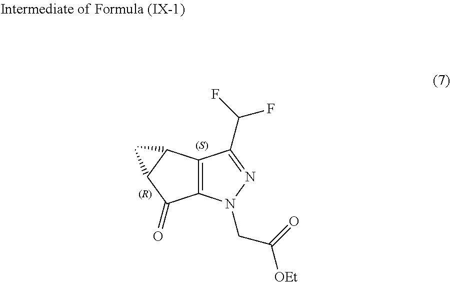

Ethyl 2-((3bS,4aS)-3-(difluoromethyl)-3b,4,4a,5-tetrahydro-1H-cyclopropa[3,4]cyclopenta[1,2-c]pyrazol-1-yl)acetate (6) (51.2 g, 1 wt., 1.0 eq.) and N-hydroxyphthalimide (HOPht, 0.1 eq.) were charged to a reaction vessel. ACN (7 vol) was added and the mixture was heated to 60° C. A solution of NaClO₂ (1.8 eq.) in water (4.3 vol) was added dropwise over 3 hours at 55° C.-65° C. and the mixture was stirred for 2-6 hours until reaction completion. The mixture was cooled to 20-30° C. and a 20% aqueous NaHSO₃ solution (3 vol) was added whilst maintaining the temperature below 30° C. for at least 30 minutes. (A starch potassium iodide paper test was conducted-result: negative). EtOAc (4 vol) was added, followed by separation of the phases and extraction of the aqueous phase with EtOAc (5 vol). The combined organic phases were washed with saturated aqueous NaHCO₃ solution (6×5 vol), then saturated aqueous Na₂SO₄ solution (5 vol). The solvent was evaporated in vacuo to give a yellow residue. IPA (0.5 vol) was charged and to the residue and the contents were heated to 50° C. MCH (7 vol) was charged at 45-55° C. and the contents stirred for 1-3 hours. The mixture was filtered at 50° C. The filtrate was evaporated in vacuo to give a yellow residue. IPA (1 vol) was charged to the residue and contents were heated to 50° C. MCH (7 vol) was charged and the contents were stirred for 1-3 hours. The contents were cooled to 0° C. and stirred for 1-10 hours. The mixture was filtered at 0° C., the cake was washed with MCH (1.5 vol) and further dried in vacuo to give (ethyl 2-(3-(difluoromethyl)-5-oxo-3b,4,4a,5-tetrahydro-1H-cyclopropa[ 3,4]cyclopenta[1,2-c]pyrazol-1-yl)acetate) (7) as a white solid (43.9 g, 98.1% chemical purity, 81.8 yield).

¹H NMR (400 MHz, CDCl₃-d) δ ppm 6.51-6.87 (m, 1H), 4.96 (s, 2H), 3.63 (t, J=6.64 Hz, 1H) 2.81 (dt, J=6.58, 4.21 Hz, 1H), 2.59 (dt, J=8.55, 4.21 Hz, 1H), 1.49-1.77 (m, 2H), 1.30 (t, J=7.14 Hz, 3H), 0.96 (t, J=7.38 Hz, 1H).

### Step 4: Synthesis of Intermediate of Formula (VII-1) (ethyl 2-(3-(difluoromethyl)-4,4a-dihydrospiro[cyclopropa[3,4]cyclopenta[1,2-c]pyrazole-5,2′-[1,3]dithiolane]-1 (3bH)-yl)acetate) (9)

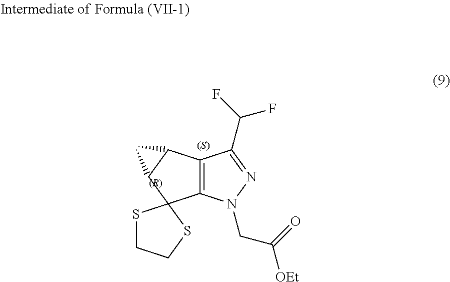

To a solution of (ethyl 2-(3-(difluoromethyl)-5-oxo-3b,4,4a,5-tetrahydro-1H-cyclopropa[3,4]cyclopenta[1,2-c]pyrazol-1-yl)acetate) (7) (43.6 g, 1 wt., 1.0 eq.) and ethane-1,2-dithiol (8) (1.2 eq.) in DCM (10 vol), BF3.2AcOH (3.0 eq.) was added at −5-5° C. The mixture was stirred at 0-10° C. for 40 hours until reaction completion. Aqueous KHCO₃ solution (15.3 wt) was added, the biphasic mixture was filtered through diatomite and the filter cake was washed with DCM (2.0 wt.). The filtrate was separated, and the organic phase was concentrated and switched with EtOH (2×3 vol), then further diluted with EtOH (3 vol). Water (9 vol) was added and the mixture was cooled and stirred, then filtered and the wet cake was dried in vacuo to give ethyl2-(3-(difluoromethyl)-4,4a-dihydrospiro[cyclopropa[3,4]cyclopenta[1,2-c]pyrazole-5,2′-[1,3]dithiolane]-1 (3bH)-yl)acetate) (9) as a solid (52.4 g, 98.0% chemical purity and 93.6% assay yield). 1H NMR (400 MHz, DMSO-d₆) δ ppm 6.75-7.14 (m, 1H), 4.84-5.13 (m, 2H), 4.14 (qd, J=7.17, 0.98 Hz, 2H), 3.38-3.67 (m, 4H), 2.66 (ddd, J=8.31, 5.62, 4.16 Hz, 1H), 2.28-2.45 (m, 1H), 1.09-1.39 (m, 4H), 0.30-0.46 (m, 1H)

### Step 5: Synthesis of Intermediate of Formula (IV-b1) (ethyl 2-(3-(difluoromethyl)-5,5-difluoro-3b,4,4a,5-tetrahydro-1H-cyclopropa[3,4]cyclopenta[1,2-c]pyrazol-1-yl)acetate) (11)

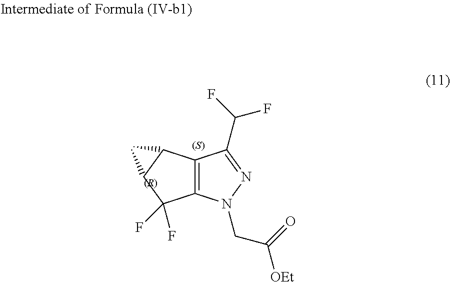

To a suspension of DBDMH (10) (3.1 eq.) in DCM (7 vol) was added dropwise at −15~−5° C. TEA.3HF (13.3 eq.). Then a solution of ethyl 2-(3-(difluoromethyl)-4,4a-dihydrospiro[cyclopropa[3,4]cyclopenta[1,2-c]pyrazole-5,2′-[1,3]dithiolane]-1 (3bH)-yl)acetate) (9) (51.9 g, 1 wt., 1.0 eq.) in DCM (3 vol) was added into the mixture at −15~−5° C. The mixture was stirred at −5-5° C. until reaction completion. A 15% aqueous Na₂SO₃ solution (7 wt.) was added at −5-5° C., the mixture was filtered through diatomite and washed with DCM. The combined filtrate was separated. The aqueous phase was extracted with DCM (3 vol). The combined organic phases were basified with 20% aqueous KHCO₃ solution (2.7 wt.). The biphasic mixture was separated and the organic phase was washed with water (2 vol). The organic phase was concentrated in vacuo and the solvent switched with THF to give (ethyl 2-(3-(difluoromethyl)-5,5-difluoro-3b,4,4a,5-tetrahydro-1H-cyclopropa[3,4]cyclopenta[1,2-c]pyrazol-1-yl)acetate) (11) in THF solution (51.9 g, after workup, 166.7 g in THE solution, 89.3% chemical purity and 23.8% assay in 90.5% assay yield). ¹H NMR (400 MHz, CDCl₃-d) δ ppm 6.47-6.89 (m, 1H), 4.85 (s, 2H), 4.26 (q, J=7.14 Hz, 2H), 2.32-2.61 (m, 2H), 1.33-1.47 (m, 1H), 1.29 (t, J=7.14 Hz, 3H), 1.15 (dtd, J=5.94, 3.92, 3.92, 2.46 Hz, 1H). 19F NMR (376 MHz, CDCl₃-d) δ ppm −112.27, −104.11, −81.28.

### Step 6: Synthesis of Intermediate of Formula (VI-b1) (2-(3-(difluoromethyl)-5,5-difluoro-3b,4,4a,5-tetrahydro-1H-cyclopropa[3,4]cyclopenta[1,2-c]pyrazol-1-yl) acetic acid) (12)

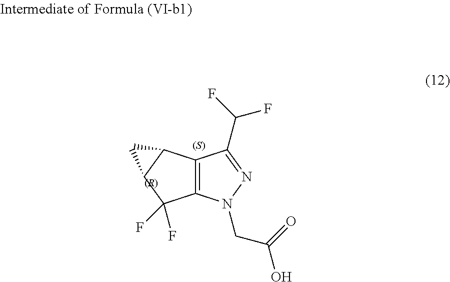

To a solution of (ethyl 2-(3-(difluoromethyl)-5,5-difluoro-3b,4,4a,5-tetrahydro-1H-cyclopropa[3,4]cyclopenta[1,2-c]pyrazol-1-yl)acetate) (11) (95.4 g, 1 wt., 1.0 eq.) in THF at 0~10° C. was added a solution of LiOH·H₂O (1.6 eq.) in water (3.5 vol). HCl was added to adjust to pH=5.5-6.5 at 10-20° C. The mixture was concentrated in vacuo below 35° C. to remove THF. Water (6 vol) was added to dissolve the residue completely at 15-20° C., then 2N HCl was added to adjust to pH=1-2 at 10-20° C. After stirring for 1 hour the reaction mixture was filtered and the cake was washed with water (2×2 vol). The wet cake was dissolved in ACN (8-10 vol) at 15-25° C., then filtered and the cake was washed with ACN (1-1.5 vol). After cycled through CUNO with active carbon and wash with ACN (2-3 vol), the filtrate was concentrated in vacuo and the solvent was switched with water. After stirring for 2-3 hours at 2-7° C., the mixture was filtered and the cake was washed with water (0.5-1 vol). The wet cake was dried in vacuo at 45-50° C. to afford (2-(3-(difluoromethyl)-5,5-difluoro-3b,4,4a,5-tetrahydro-1H-cyclopropa[3,4]cyclopenta[1,2-c]pyrazol-1-yl) acetic acid) (12) as a yellow solid. Repurification: The solid was taken up in EtOAc, cycled through CUNO with active carbon and washed with further EtOAc. The solvent was switched with ACN, then cycled through CUNO with active carbon and washed with ACN. The filtrate was concentrated in vacuo and the solvent switched with water and filtered. The cake was washed with water and further dried in vacuo to afford (2-(3-(difluoromethyl)-5,5-difluoro-3b,4,4a,5-tetrahydro-1H-cyclopropa[3,4]cyclopenta[1,2-c]pyrazol-1-yl) acetic acid) (12) as a yellow solid (66.6 g, 99.6% chemical purity, 99.9% chiral purity, 77.3% assay yield).

¹H NMR (400 MHz, DMSO-d₆) δ ppm 13.17-13.60 (m, 1H), 6.79-7.27 (m, 1H), 4.76-5.04 (m, 2H), 2.50-2.71 (m, 2H), 1.44 (qd, J=6.93, 1.11 Hz, 1H), 0.89-1.02 (m, 1H).

### Example 2

### Step A: Preparation of Intermediate of Formula (I-a2) ((1S,5S)-2-(2,2,2-trifluoroacetyl) bicyclo[3.1.0]hexan-3-one) (A)

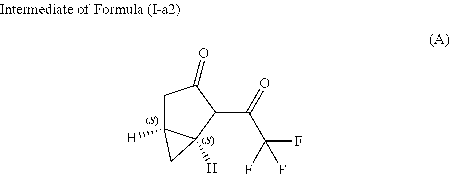

(R)-bis((R)-1-phenylethyl)amine hydrochloride (2) (1.1 eq.) was charged to a reaction vessel equipped with stirrer and internal thermometer. THF (31 mL) was added, followed by LiCl (57.2 mL, 1.1 eq.) in THF (0.5 M), then the reaction vessel was degassed and purged with nitrogen for 3 times. The vessel was cooled in an acetone/dry ice bath until internal reading reached-65~−75° C. n-BuLi (11.4 mL, 2.2 eq., 2.5M) was added dropwise whilst maintaining the internal temperature <−60° C. The contents were stirred at −65~−75° C. for 10 minutes then warmed to 20° C. over 1 hour. The contents were then cooled back to −65~−75° C. In a separate vessel a solution of bicyclo[3.1.0]hexan-3-one (1) (2.5 g, 1 wt., 1.0 eq.) in THF (12.0 mL) was prepared and the vessel was purged with nitrogen for 10-15 minute. The freshly prepared ketone solution was added dropwise to the lithium amide base solution at −65~−75° C., whilst maintaining the internal temperature <−65° C. The contents were stirred for 20 minutes at −65~−75° C. Next ethyl 2,2,2-trifluoroacetate (X) (3.71 mL, 1.2 eq.) was added dropwise at −65~−75° C., whilst maintaining the internal temperature <−65° C. The contents were stirred at −65~−75° C. for 1-4 hours. The reaction was quenched with HCl (19.07 mL, 3M solution in CPME) by fast addition to the cold mixture. The mixture was then allowed to warm to 20° C. whilst stirring. Water (60 mL) and MTBE (60 mL) were added and the solution was stirred for 10 minutes. The phases were allowed to separate, then the aqueous phase was discharged. The organic phase was washed with 2M HCl (2×60 mL), then water (2×60 mL), then brine (60 mL) and the solution was concentrated in vacuo to obtain crude ((1S,5S)-2-(2,2,2-trifluoroacetyl) bicyclo[3.1.0]hexan-3-one) (A) as an oil (6.57 g), which was directly submitted to step B.

### Step B: Preparation of Intermediate of Formula (VI-a2) ((ethyl 2-((3bS,4aS)-3-(trifluoromethyl)-3b,4,4a,5-tetrahydro-1H-cyclopropa[3,4]cyclopenta[1,2-c]pyrazol-1-yl)acetate) (B)

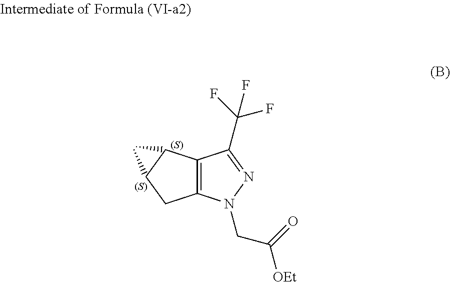

Aminoglycinate hydrochloride (5) (4.83 g, 1.2 eq.) was charged to a reaction vessel. 2-MeTHF (75 mL) was added and the vessel was purged with nitrogen for 10-15 minutes. Sulfuric acid (6.94 mL) was added dropwise at −10-0° C. In a separate vessel a solution of 2-(2,2,2-trifluoroacetyl) bicyclo[3.1.0]hexan-3-one (A) (5 g, 1 wt., 1.0 eq.) in EtOH (75 mL) was prepared. The freshly prepared solution of 2-(2,2,2-trifluoroacetyl) bicyclo[3.1.0]hexan-3-one (A) was added to the hydrazine containing reaction vessel at 0° C. The mixture was allowed to warm to 20° C. until complete consumption of starting material (by GC) was observed. Water (150 mL) and EtOAc (200 mL) were added. The phases were allowed to separate. The aqueous phase was washed with EtOAc (3×50 mL), the combined organic phases were washed with brine (2×50 mL) and concentrated to afford a crude brown oil (regioisomer ratio 3.2:1, B: regioisomer-B). The crude oil was purified by column chromatography to give ethyl 2-((3bS,4aS)-3-(trifluoromethyl)-3b,4,4a,5-tetrahydro-1H-cyclopropa[3,4]cyclopenta[1,2-c]pyrazol-1-yl)acetate (B) as a white solid (2.65 g, 90.1% chiral purity, 79% ee, enantiomer favored is unknown, 37.1% yield over step A and B).

¹H NMR (400 MHz, CDCl₃-d) δ ppm 4.63-4.84 (m, 2H), 4.23 (q, J=7.14 Hz, 2H), 2.81-2.99 (m, 1H), 2.66-2.79 (m, 1H), 2.05-2.22 (m, 2H), 1.28 (t, J=7.14 Hz, 3H), 1.12 (td, J=7.75, 5.17 Hz, 1H), 0.26-0.40 (m, 1H). ¹³C NMR (101 MHz, CDCl₃-d) δ ppm 166.8, 151.3, 13.9, 134.8-136.4 (m, 1 C), 129.8, 121.4 (q, J=268.55 Hz, 1 C), 62.1, 52.2, 27.3, 21.8, 17.40, 14.1. 19F NMR (376 MHz, CDCl₃-d) δ ppm-61.63. MS (ESI): m/z [M+H+] calcd for C₁₂H₁₄F₃N₂O₂: 275.0929; found: 275.1020.

### Step C: Preparation of Intermediate of Formula (I-a3) ((1S,5S)-2-(cyclopropanecarbonyl) bicyclo[3.1.0]hexan-3-one) (C)

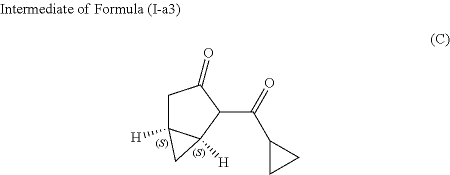

(R)-bis((R)-1-phenylethyl)amine hydrochloride (2) (1.1 eq.) was charged to a reaction vessel equipped with stirrer and internal thermometer. THF (25 mL) was added, followed by LiCl (45.8 mL, 1.1 eq.) in THF (0.5 M), then the reaction vessel was degassed and purged with nitrogen for 3 times. The vessel was cooled in an acetone/dry ice bath until internal reading reached-65~−75° C. n-BuLi (9.15 mL, 2.2 eq., 2.5M) was added dropwise whilst maintaining the internal temperature <−60° C. The contents were stirred at −65~−75° C. for 10 minutes then warmed to 20° C. over 1 hour. The contents were then cooled back to −65~−75° C. In a separate vessel a solution of bicyclo[3.1.0]hexan-3-one (1) (10.4 mL, 1 wt., 1.0 eq.) in THF (10.0 mL) was prepared and the vessel was purged with nitrogen for 10-15 minute. The freshly prepared ketone solution was added dropwise to the lithium amide base solution at −65~−75° C., whilst maintaining the internal temperature <−65° C. The contents were stirred for 20 minutes at −65~−75° C. Next cyclopropanecarbonyl chloride (Y) (2.7 mL) was added dropwise at −65~−75° C., whilst maintaining the internal temperature <−65° C. The contents were stirred at −65~−75° C. for 1-4 hours. The reaction was quenched with HCl (15.3 mL, 3M solution in CPME) by fast addition to the cold mixture. The mixture was then allowed to warm to 20° C. whilst stirring. Aqueous 2M HCl (50 mL) and MTBE (50 mL) were added and the solution was stirred for 10 minutes. The phases were allowed to separate, then the aqueous phase was discharged. The organic phase was washed with 2M HCl (2×50 mL), then water (2×50 mL), then brine (30 mL), filtered through a hydrophobic frit and the solvent was evaporated in vacuo to obtain crude ((1S,5S)-2-(2,2,2-trifluoroacetyl) bicyclo[3.1.0]hexan-3-one) (C) as an oil (4.00 g), which was directly submitted to step D.

### Step D: Preparation of Intermediate of Formula (VI-a3) (ethyl 2-((3bS,4aS)-3-cyclopropyl-3b,4,4a,5-tetrahydro-1H-cyclopropa[3,4]cyclopenta[1,2-c]pyrazol-1-yl)acetate) (D)

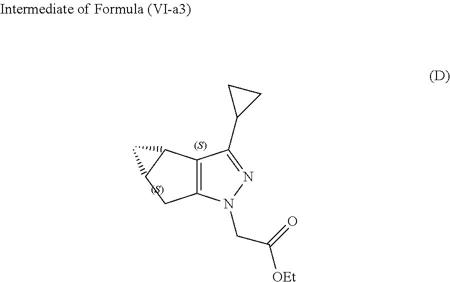

Aminoglycinate hydrochloride (5) (3.86 g, 1.2 eq.) was charged to a reaction vessel. 2-MeTHF (50 mL) was added and the vessel was purged with nitrogen for 10-15 minutes. The mixture was cooled to −10-0° C. Sulfuric acid (5.55 mL) was added dropwise. In a separate vessel a solution of 2-(cyclopropanecarbonyl) bicyclo[3.1.0]hexan-3-one (3.42 g, 1 wt., 1.0 eq.) in EtOH (50 mL) was prepared. The freshly prepared solution of 2-(cyclopropanecarbonyl) bicyclo[3.1.0]hexan-3-one was added to the hydrazine containing reaction vessel at 0° C. The mixture was allowed to warm to 20° C. until complete consumption of starting material (by GC) was observed. Water (100 mL) and EtOAc (150 mL) were added. The phases were allowed to separate. The aqueous phase was washed with EtOAc (3×30 mL), the combined organic phases were washed with brine (2×30 mL) and concentrated in vacuo to afford a brown oil. The oil was taken up in EtOAc (100 mL) and washed with water (15 mL), saturated aqueous NaHCO₃ solution (40 mL), water (40 mL) and brine (20 mL). The organic phase was concentrated in vacuo to give 2.18 g of a crude oil (42.5% crude yield over step A and B, 60.7% purity, regioisomer ratio 2.5:1, D: regiosiomer-D). A proportion (0.1 g) of the crude oil was purified by mass directed auto purification (MDAP; water/ACN, formic additive), followed by an aqueous work-up with EtOAc (50 mL) and saturated aqueous NaHCO₃ solution (10 mL). Evaporation in vacuo gave (ethyl2-((3bS,4aS)-3-cyclopropyl-3b,4,4a,5-tetrahydro-1H-cyclopropa[ 3,4]cyclopenta[1,2-c]pyrazol-1-yl)acetate) (D) as a white solid (0.03 g, 87.5% chiral purity, 75% ee, enantiomer favoured is unknown).

¹H NMR (400 MHz, CDCl₃-d) δ ppm 4.54-4.70 (m, 2H, CH₂), 4.18-4.28 (m, 2H, CH₂CH₃), 2.79-2.87 (m, 1H, CH₂CH), 2.57-2.67 (m, 1H, CH₂CH), 1.95-2.03 (m, 1H, CHa), 1.83-1.95 (m, 2H, CH₂CHCH₂ & CHb), 1.23-1.32 (m, 3H, CH₂CH₃), 0.97-1.05 (m, 1H, CHCH₂CH), 0.75-1.04 (m, 4H, CH₂CH₂), 0.17-0.26 (m, 1H, CHCH₂CH). 13C NMR (101 MHz, CDCl₃-d) δ ppm 168.0, 150.3, 148.8, 126.8, 61.6, 51.4, 26.7, 21.3, 17.5, 14.1, 13.8, 9.1, 7.3, 6.8. MS (ESI): m/z [M+H+] calcd for C₁₄H₁₉N₂O₂:247.1368; found: 247.1450.

### Example 3

### Preparation of 2-((3bS,4aS)-3-cyclopropyl-3b,4,4a,5-tetrahydro-1H-cyclopropa[3,4]cyclopenta[1,2-c]pyrazol-1-yl) acetic acid

### Synthetic Scheme

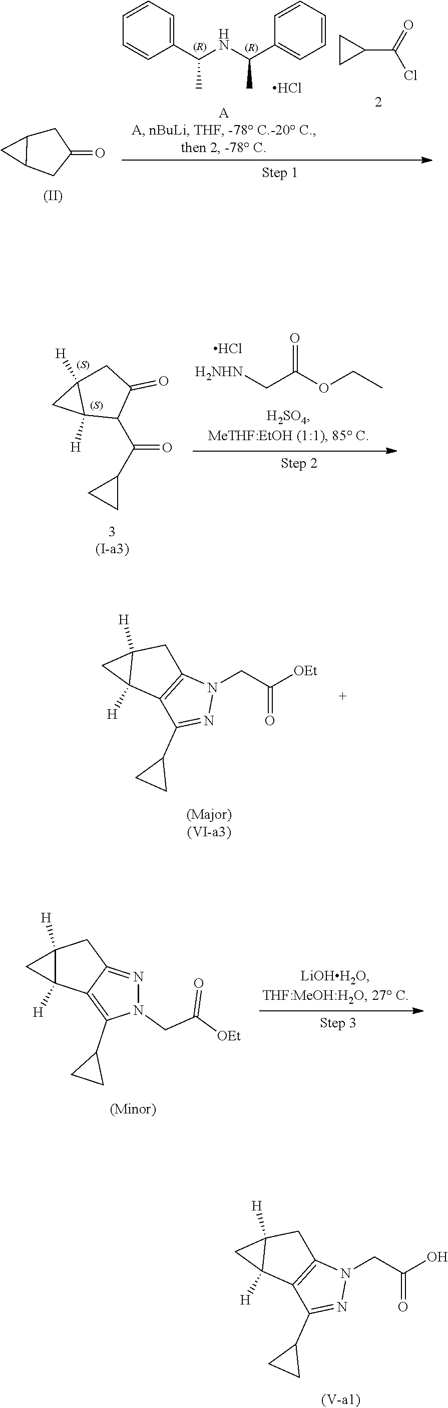

### Step 1: Preparation of Intermediate of Formula (I-a3) (1S,5S)-2-(cyclopropanecarbonyl) bicyclo[3.1.0]hexan-3-one

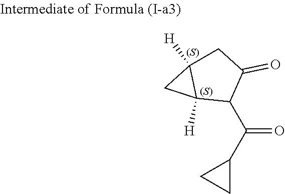

To a stirred solution of (R)-bis((R)-1-phenylethyl)amine HCl salt (1263 mg, 5.61 mmol) in Tetrahydrofuran (THF) (2 mL) was added anhydrous lithium chloride (238 mg, 5.61 mmol) under nitrogen atmosphere. The reaction mixture was cooled to −78° C., then n-butyl lithium (2.5 M in Hexanes, 4.49 mL, 11.21 mmol) was added drop wise at −78° C. The solution was stirred for 10 minutes and then was allowed to warm to 20° C. The mixture was stirred for 1 h. The reaction mixture was again cooled to −78° C. and then to the mixture was added a solution of bicyclo[3.1.0]hexan-3-one (0.437 mL, 5.10 mmol) in tetrahydrofuran (THF) (2 mL). The mixture was stirred for 20 minutes. Cyclopropane carbonyl chloride (0.510 mL, 5.61 mmol) was added to the mixture drop wise at −78° C. and then the mixture was stirred at same temperature for 4 h. The progress of the reaction was monitored by TLC (SiO₂, 10% EtOAc/Pet-ether, Rf=0.4, PMA active). On completion, the reaction mixture was quenched with 4M HCl in ethyl acetate (4 mL), 2N aqueous HCl solution (5 mL) and MTBE (10 mL) and the resulting mixture was stirred for 20 min. The mixture was filtered and the filtrate was partitioned. The aqueous layer was extracted with ethyl acetate (10 mL). The combined organic layers were washed with 2N aqueous HCl solution (2×5 mL) and water (2×5 mL). The organic layer was washed with brine (5 mL) and dried over anhydrous Na₂SO₄, filtered and the filtrate was concentrated under reduced pressure to afford crude (1S,5S)-2-(cyclopropanecarbonyl) bicyclo[3.1.0]hexan-3-one (870 mg) as a pale-yellow liquid. The product was used directly in the next step. LCMS Method=Column: ACQUITY UPLC BEH C18 1.7 μm, 2.1 x 50 mm; Mobile Phase C: 0.05% formic acid in water, Mobile Phase D: 0.05% formic acid in MeCN; Gradient (minute/% D): 0/3, 0.4/3, 2.5/98, 3.4/98, 3.5/3, 4.5/3; Flow-rate: 0.6 mL/min; Temp: 40° C. LCMS result: retention time=2.31 mins; observed ion=165.06 (M+H).

### Step 2: Preparation of Intermediate of Formula (VI-a3) (ethyl 2-((3bS,4aS)-3-cyclopropyl-3b,4,4a,5-tetrahydro-1H-cyclopropa[3,4]cyclopenta[1,2-c]pyrazol-1-yl)acetate)

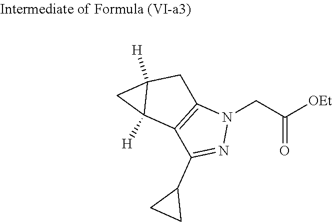 [SMILES: CCOC(=O)Cn1nc(C2CC2)c2c1C[C@@H]1C[C@H]21]

To a stirred solution of 2-(cyclopropanecarbonyl) bicyclo[3.1.0]hexan-3-one (870 mg, 3.97 mmol, see previous step) in ethanol (10 mL), 2-methyltetrahydrofuran (2-MeTHF) (10 mL) and sulfuric acid (0.087 mL, 1.641 mmol) was added ethyl aminoglycinate hydrochloride (737 mg, 4.77 mmol) portion wise at 10° C. under N₂ atmosphere. The reaction mixture was stirred at 85° C. for 16 h. The progress of the reaction was monitored by TLC (SiO₂, 20% Acetone/Pet.ether; Rf=0.3, UV active). On completion, the reaction mixture was concentrated under reduced pressure and the resulting residue was diluted with ethyl acetate (50 mL) and then washed with water (30 ml). The aqueous layer was extracted with ethyl acetate (2×50 mL). The combined organic layers were washed with brine (20 mL) and dried over anhydrous Na₂SO₄, filtered and the filtrate was concentrated under reduced pressure to afford the crude product (1 g) as a brown liquid. The crude product was subjected to silica gel chromatography (12 g, Flashpure-Buchi cartridge) eluting with a gradient of 10-20% EtOAc in petroleum ether to afford the enriched product ethyl 2-(3-cyclopropyl-3b,4,4a,5-tetrahydro-1H-cyclopropa[3,4]cyclopenta[1,2-c]pyrazol-1-yl)acetate (400 mg, LCMS purity 33%) as a mixture of regioisomers as a pale yellow liquid. The material was then was adsorbed onto celite and the resulting powder was subjected to C18 chromatography using a Combi-Flash system equipped with a RediSep Gold C18 column (40 g, Teledyne Isco) eluting with a gradient of 50-65% MeCN (containing 0.1% TFA) in water (containing 0.1% TFA) with flow rate 20 mL/minute. The pure product-containing fractions were combined and concentrated under reduced pressure. The resulting aqueous solution was adjusted to pH~7 by the addition of aqueous saturated NaHCO₃ solution. The solution was extracted with EtOAc (3×50 mL), dried over Na₂SO₄, filtered, and the filtrate was concentrated under reduced pressure to afford enriched ethyl 2-(3-cyclopropyl-3b,4,4a,5-tetrahydro-1H-cyclopropa[3,4]cyclopenta[1,2-c]pyrazol-1-yl)acetate (210 mg, 15% yield over in two steps) as pale yellow gummy oil, a mixture of the two regioisomers. The material was used directly in the next step without further purification. ¹H NMR (400 MHz, CDCl₃) 0=4.68-4.53 (m, 2H), 4.24-4.18 (m, 2H), 2.83-2.76 (m, 1H), 2.67-2.58 (m, 1H), 1.96-1.83 (m, 3H), 1.29-1.21 (m, 3H), 1.06-0.73 (m, 5H), 0.21-0.16 (m, 1H). LCMS Method=Column: CORTECS UPLC C18 1.6 μm, 3.0×30 mm; Mobile phase A: 0.05% formic acid in Water, Mobile Phase B: 0.05% formic acid in MeCN; Gradient (minute/% B): 0/3, 0.1/3, 1.2/98, 2.0/98, 2.05/3, 2.50/3; Flow-rate: 0.85 mL/min; Temp: 45° C. LCMS result: retention time=1.10 mins.; observed ion=247.56 (M+H). LCMS Purity=88%, HPLC: purity: 64.8%, 21.9%, RT=3.68, 4.15 min.

### Step 3: Preparation of Intermediate of Formula (V-a1) (2-((3bS,4aS)-3-cyclopropyl-3b,4,4a,5-tetrahydro-1H-cyclopropa[3,4]cyclopenta[1,2-c]pyrazol-1-yl) acetic acid)

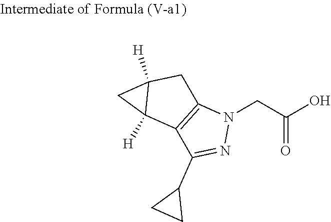 [SMILES: O=C(O)Cn1nc(C2CC2)c2c1C[C@@H]1C[C@H]21]

To a stirred solution of ethyl 2-(3-cyclopropyl-3b,4,4a,5-tetrahydro-1H-cyclopropa[3,4]cyclopenta[1,2-c]pyrazol-1-yl)acetate (210 mg, 0.758 mmol, see previous step) in tetrahydrofuran (THF) (2 mL) and methanol (1 mL) was added at 0° C. a solution of LiOH·H₂O (91 mg, 3.79 mmol) dissolved in Water (1 mL). The reaction mixture was allowed to warm to 27° C. and was then stirred for 2 h. The progress of the reaction was monitored by TLC (SiO₂, 10% MeOH in DCM. Rf=0.1, UV active). On completion, the organic solvents were evaporated under reduced pressure. The pH of the resulting aqueous layer was adjusted to ~2 by the addition of 1N aqueous HCl solution (2 mL). The precipitated solid was collected by filtration and then washed with water (5 L). The solids were triturated with n-pentane (5 mL) and then dried under vacuum to afford 2-(3-cyclopropyl-3b,4,4a,5-tetrahydro-1H-cyclopropa[3,4]cyclopenta[1,2-c]pyrazol-1-yl) acetic acid (120 mg) as a mixture of regioisomers and enantiomers. LCMS Method=Column: CORTECS UPLC C18 (30×3 mm, 1.6 μm); Mobile Phase A: 0.05% Formic Acid in water; Mobile Phase B: 0.05% Formic Acid in MeCN; Time (min)/% B: 0/3, 0.1/3, 1.2/98, 2/98, 2.05/3, 2.5/3; Column Temp: 45° C.; Flow Rate: 0.85 ml/min. LCMS Result: retention time=0.87 & 0.92 min; observed ion=219.16 (M+H); LCMS Purity=54% & 27% (mixture of regioisomers). Chiral SFC method=Column: CHIRALPAK IC (4.6×150 mm) 5 μm; Co-solvent: 0.5% triethylamine in methanol; Total flow: 3 mL/min; Eluent: 70% CO₂, 30% co-solvent; ABPR: 1500 psi; Column Temperature: 30° C.; Detection: Spectrum PDA 237.0 nm. Note: Co-injection of an authentic homochiral sample of 2-((3bS,4aS)-3-cyclopropyl-3b,4,4a,5-tetrahydro-1H-cyclopropa[3,4]cyclopenta[1,2-c]pyrazol-1-yl) acetic acid was used to confirm the identity of the desired product peak. Chiral SFC Result: Purity % (retention time; identity): 9.37% (2.82 min; 2-((3bR,4aR)-3-cyclopropyl-3b,4,4a,5-tetrahydro-1H-cyclopropa[3,4]cyclopenta[1,2-c]pyrazol-1-yl) acetic acid), 29.41% (3.31 min, 2-(3-cyclopropyl-3b,4,4a,5-tetrahydro-2H-cyclopropa[3,4]cyclopenta[1,2-c]pyrazol-2-yl) acetic acid, stereochemistry unassigned), 56.32% (4.25 min, 2-((3bS,4aS)-3-cyclopropyl-3b,4,4a,5-tetrahydro-1H-cyclopropa[3,4]cyclopenta[1,2-c]pyrazol-1-yl) acetic acid), 4.9% (6.02 min, 2-(3-cyclopropyl-3b,4,4a,5-tetrahydro-2H-cyclopropa[3,4]cyclopenta[1,2-c]pyrazol-2-yl) acetic acid, stereochemistry unassigned). Summary: 2:1 ratio of regioisomeric mixture and major component is the title compound; 71% enantiomeric excess and major component is the title compound (S,S stereochemistry).

### Example 4

### 5 Preparation of 2-((3bS,4aS)-3-cyclopropyl-3b,4,4a,5-tetrahydro-1H-cyclopropa[3,4]cyclopenta[1,2-c]pyrazol-1-yl) acetic acid

### Synthetic Scheme

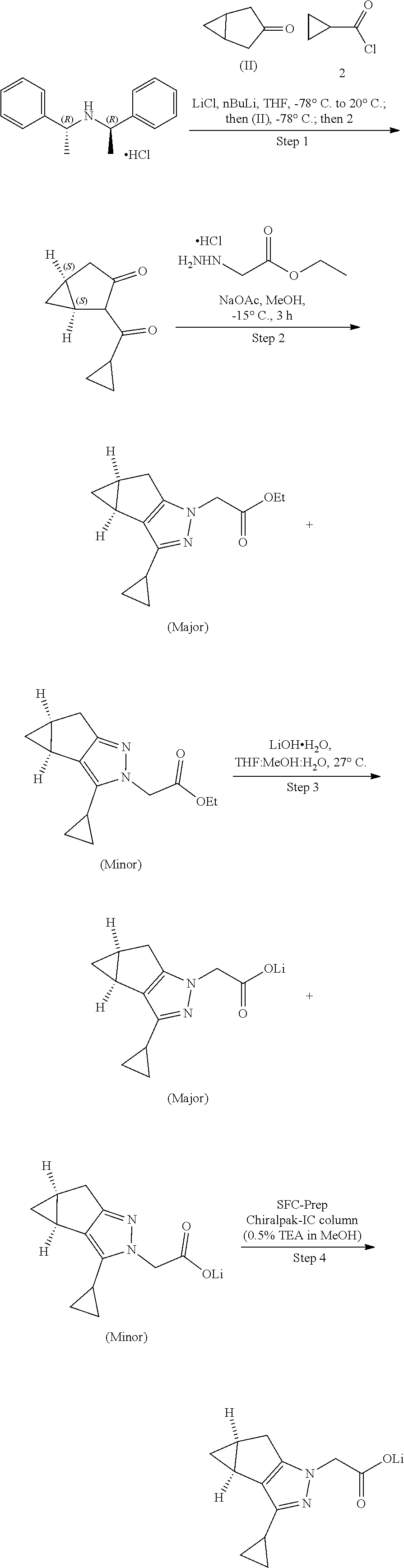

### Step 1: Preparation of Intermediate of Formula (I-a3) ((1S,5S)-2-(cyclopropanecarbonyl) bicyclo[3.1.0]hexan-3-one)

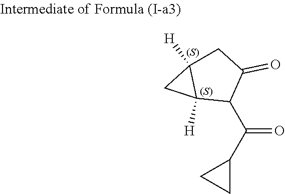

To a stirred solution of (R,R)-bis(alpha-methylbenzyl)amine hydrochloride (CAS: 82398-30-9) (294 g, 1.12 mol, 1.1 eq) in tetrahydrofuran (THF) (1 L) under nitrogen atmosphere was added anhydrous lithium chloride (47.5 g, 1.12 mol). The reaction mixture was cooled to −78° C. To the mixture at −78° C. was slowly added n-butyllithium (2.5 M solution in hexanes; 897 mL, 2.24 mol). The mixture was then stirred at 20° C. for 1 h. The reaction mixture was cooled to −78° C. and to the mixture was added over 20 minutes a solution of bicyclo[3.1.0]hexan-3-one (100 g, 1.019 mol) in tetrahydrofuran (100 mL). To the mixture at −78° C. was added dropwise cyclopropane carbonyl chloride (101.8 mL, 1.121 mol). The mixture was stirred for 3 h, then the reaction mixture was quenched by the addition of 2N aqueous HCl solution (500 mL). The mixture was stirred for 20 min. The mixture was filtered, and the filter pad was extracted with pet. ether (2×500 mL). The filtrate was partitioned and the isolated organic layer was washed with water (2×500 mL), then brine (2×500 mL). The organic phase was dried over anhydrous Na₂SO₄, filtered, and then concentrated under reduced pressure to afford crude (1S,5S)-2-(cyclopropanecarbonyl) bicyclo[3.1.0]hexan-3-one (160 g) as a grey color liquid. This crude compound was used directly in the next step. LCMS Method: Column=ACQUITY UPLC BEH C18 1.7 μm (2.1×50 mm); Mobile Phase A=0.05% formic acid in water; Mobile Phase B=0.05% formic acid in acetonitrile; Gradient (minutes/% B)=0/3, 0.4/3, 2.5/98, 3.4/98, 3.5/3, 4.5/3; Flow rate=0.6 mL/min; Column Temp=40° C. LCMS Result: retention time=2.13 min; observed ion=164.99 [M+H]+; LCMS Purity=56.7%.

### Step 2: Preparation of Intermediate of Formula (VI-a3) (ethyl 2-((3bS,4aS)-3-cyclopropyl-3b,4,4a,5-tetrahydro-1H-cyclopropa[3,4]cyclopenta[1,2-c]pyrazol-1-yl)acetate)

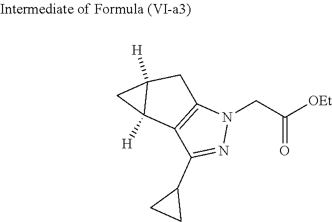 [SMILES: CCOC(=O)Cn1nc(C2CC2)c2c1C[C@@H]1C[C@H]21]

To a stirred solution of (1S,5S)-2-(cyclopropanecarbonyl) bicyclo[3.1.0]hexan-3-one (160 g, 56% wt, 546 mmol, from previous step) in methanol (2.4 L) at −15° C. under N₂ atmosphere was added ethyl aminoglycinate hydrochloride (122 g, 789 mmol) and sodium acetate (163 g, 1.99 mol). The mixture was stirred at −15° C. to −10° C. for 3 h. The reaction mixture was concentrated under reduced pressure and the resulting residue was diluted with water (2 L) and then extracted with ethyl acetate (2 X 1 L). The combined organics were washed with brine (1 L), dried over anhydrous Na₂SO₄, filtered, and the filtrate was concentrated under reduced pressure to afford the crude product (200 g) as a brown gum. The crude compound was adsorbed onto silica gel and the resulting powder was subjected to silica gel chromatography (100-200 mesh) eluting with a gradient of 13-40% ethyl acetate in pet. ether to afford ethyl 2-((3bS,4aS)-3-cyclopropyl-3b,4,4a,5-tetrahydro-1H-cyclopropa[3,4]cyclopenta[1,2-c]pyrazol-1-yl)acetate (85 g, Yield=33%), a mixture of regioisomers and enantiomers, as a brown gum. ¹H NMR (400 MHz, DMSO-d6) δ=4.94-4.81 (m, 2H (minor)), 4.70 (s, 2H (major)), 4.21-4.11 (m, 2H (major)), 4.11-4.02 (m, 2H (minor)), 2.75-2.65 (m, 1H (major+minor)), 2.61-2.51 (m, 1H (major+minor)), 2.01-1.82 (m, 2H (major+minor)), 1.81-1.71 (m, 1H (major+minor)), 1.23-1.16 (m, 3H (major+minor)), 1.02-0.75 (m, 3H (major+minor)), 0.72-0.59 (m, 2H (major+minor)), 0.08-−0.03 (m, 1H (major+minor)). LCMS Method: Column=ACQUITY UPLC BEH C18 1.7 μm (2.1×50 mm); Mobile Phase A=0.05% formic acid in water; Mobile Phase B=0.05% formic acid in acetonitrile; Gradient (minutes/% B)=0/3, 0.4/3, 7.5/98, 9.5/98, 9.6/3, 10/3; Flow rate=0.6 mL/min; Column Temp=40° C. LCMS result: retention time=3.92, 4.05 mins.; observed ion=247.02 [M+H [⁺; LCMS Purity=51.9% & 27.8%. HPLC: retention time=3.07, 3.48 min, HPLC Purity=77.2% & 16.5%.

### Step 3: Preparation of the lithium salt of Intermediate of Formula (V-a1) (lithium 2-((3bS,4aS)-3-cyclopropyl-3b,4,4a,5-tetrahydro-1H-cyclopropa[3,4]cyclopenta[1,2-c]pyrazol-1-yl)acetate)

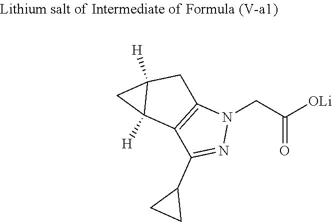

To a stirred solution of ethyl 2-((3bS,4aS)-3-cyclopropyl-3b,4,4a,5-tetrahydro-1H-cyclopropa[3,4]cyclopenta[1,2-c]pyrazol-1-yl)acetate (160 g, 65% Wt, 422 mmol) in tetrahydrofuran (700 mL) and methanol (300 mL) at 0° C. was added a solution of lithium hydroxide monohydrate (62.0 g, 1.48 mol) in water (150 mL). The reaction mixture was stirred at 0° C. to 10° C. for 1 h. The reaction mixture was concentrated under reduced pressure and the resulting crude residue was acidified with 1N aqueous HCl solution (200 mL) to a pH or ~5-6 and the mixture was stirred for 10 min. The solid precipitate was collected by filtration, washed with water (100 mL) and n-pentane (200 mL), and then dried under vacuum to afford the title compound, lithium 2-((3bS,4aS)-3-cyclopropyl- 3b,4,4a,5-tetrahydro-1H-cyclopropa[3,4]cyclopenta[1,2-c]pyrazol-1-yl)acetate (68 g, HPLC: 98% purity, Chiral SFC: 88.7% purity) as an off white solid. ¹H NMR (400 MHz, DMSO-d6) δ=4.02 (q, 2H, J=10.4 Hz), 2.75-2.65 (m, 1H), 2.54-2.50 (m, 1H), 1.89-1.86 (m, 1H), 1.82-1.79 (m, 1H), 1.73-1.69 (m, 1H), 0.92-0.88 (m, 1H), 0.79-0.74 (m, 2H), 0.65-0.62 (m, 2H), 0.11-0.08 (m, 1H). LCMS Method: Column=ACQUITY UPLC BEH C18 1.7 μm (2.1×50 mm); Mobile Phase A=0.05% formic acid in water; Mobile Phase B=0.05% formic acid in acetonitrile; Gradient (minutes/% B)=0/3, 0.4/3, 7.5/98, 9.5/98, 9.6/3, 10/3; Flow rate=0.6 mL/min; Column Temp=40° C. LCMS Result: retention time=2.66 mins.; observed ion=218.97 [M+H]+; HPLC Method=XSelect CSH C18 3.5 μm (4.6×150 mm); Mobile Phase A=0.05% trifluoroacetic acid in water; Mobile Phase B=100% acetonitrile; Gradient (minutes/% B)=0/5, 1/5, 3/15, 7/55, 11/98, 16/98, 16.1/5, 20/5; Flow rate=1.0 mL/min; Column Temp=ambient; HPLC Result: retention time=7.57 mins, Purity=98%; Chiral SFC Method: Column=CHIRALPAK IC 5 μm (2.1×50 mm); Eluent=CO₂:0.5% triethylamine in MeOH (70:30); Back pressure=1500 psi; Flow rate=4 mL/min; Column Temp=30° C. Chiral SFC Result=88.7% major peak, retention time major peak=2.96 min, retention time minor peak=2.13. Li Content=3.60% w/w (1.2 eq). Note: Analysis of the filtrate found a mixture of regioisomers (70 g, LCMS: 63% title compound, 25% regioisomer of the title compound).

### Step 4: SFC purification of the lithium salt of Intermediate of Formula (V-a1) (lithium 2-((3bS,4aS)-3-cyclopropyl-3b,4,4a,5-tetrahydro-1H-cyclopropa[3,4]cyclopenta[1,2-c]pyrazol-1-yl)acetate)

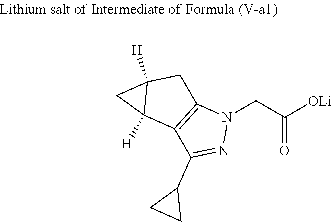

Chirally-enriched lithium 2-((3bS,4aS)-3-cyclopropyl-3b,4,4a,5-tetrahydro-1H-cyclopropa[3,4]cyclopenta[1,2-c]pyrazol-1-yl)acetate (70 g) was purified by SFC chromatography according to the following method: Column=Chiralpak IC 5μ (250×30 mm); Eluent=CO₂:0.5% triethylamine in MeOH (70:30); Flow rate=100 g/min; Back pressure=100 bar; Detection=240 nm (UV); Stack time=6.7 min; Load per injection=1.50 g. The pure major peak (Peak-2) fractions were collected and concentrated under reduced pressure to afford lithium 2-((3bS,4aS)-3-cyclopropyl-3b,4,4a,5-tetrahydro-1H-cyclopropa[3,4]cyclopenta[1,2-c]pyrazol-1-yl)acetate (45 g, LCMS: 98% purity, Chiral SFC: 99.9% purity) as an off white solid. The obtained compound was further treated with aqueous 1N HCl solution (150 mL) at 0° C. and stirred for 10 min. The solids were collected by filtration and then were washed with water (20 mL) and n-Pentane (100 mL). The solids were dried under vacuum to afford lithium 2-((3bS,4aS)-3-cyclopropyl-3b,4,4a,5-tetrahydro-1H-cyclopropa[3,4]cyclopenta[1,2-c]pyrazol-1-yl)acetate (30.08 g) as an off white solid. ¹H NMR (400 MHz, DMSO-d6) δ=4.02 (q, 2H, J=10.4 Hz), 2.75-2.65 (m, 1H), 2.54-2.50 (m, 1H), 1.89-1.86 (m, 1H), 1.82-1.79 (m, 1H), 1.73-1.69 (m, 1H), 0.92-0.88 (m, 1H), 0.79-0.74 (m, 2H), 0.65-0.62 (m, 2H), 0.11-0.08 (m, 1H). LCMS Method: Column=X Bridge C18 3.5 μm (4.6×150 mm); Mobile Phase A=10 mM Ammonium Bicarbonate in Water; Mobile Phase B=100% acetonitrile; Gradient (minutes/% B): 0/5, 1/5, 3/15, 7/55, 11/98, 16/98, 16.01/5, 20/5; Column Temp=ambient; Flow rate=1.0 mL/min. LCMS Result: retention time=6.68 mins.; observed ion=219.15 [M+H]+; LCMS Purity=99%. HPLC: retention time=6.83 mins; HPLC Purity=98%. Chiral SFC method: Column=CHIRALPAK IC 5 μm (4.6×150 mm); Eluent=CO₂:0.5% diethylamine in methanol (65:35); Total flow: =3 mL/min; Back pressure=1500 psi; Temperature=30° C. Chiral SFC result: retention time=1.78 mins; Purity: 99.8%. Specific optical rotation [α]²⁵_{D} (0.34% in Methanol)=−11.824°. Li content=3.60% w/w (1.15 eq). Water content=1.33%.

1. A process for preparing a mixture comprising a compound of formula (I-a) 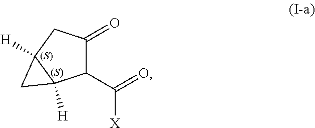
and a compound of formula (I-b) 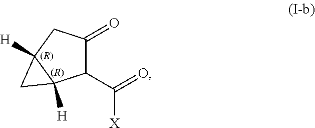
comprising combining:
(a) a compound of formula (II), 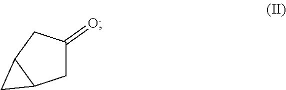
(b) a compound of formula (III), 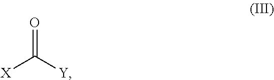
wherein X is cyclopropyl, difluoromethyl, or trifluoromethyl and Y is a leaving group;
(c) a chiral amine;
(d) a lithium base;
(e) optionally, a lithium salt; and
(f) an aprotic solvent;
to provide the mixture.

2. The process according to claim 1, wherein the chiral amine (c) is a compound selected from formula (IV-a), (IV-b), (IV-c), (IV-d), (IV-e), or (IV-f): 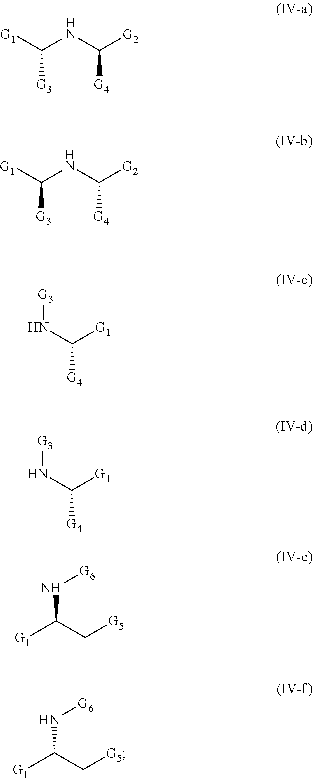
wherein
G₁ is unsubstituted phenyl or phenyl substituted by one, two, or three substituents independently selected from halogen, cyano, substituted C₁-C₄ alkyl, unsubstituted C₁-C₄ alkyl, —O (cyclopropyl), —O (allyl), allyl, —O(R₁), or —C(O) (R₁);
G₂ is unsubstituted phenyl or phenyl substituted by one, two, or three substituents independently selected from halogen, cyano, substituted C₁-C₄ alkyl, unsubstituted C₁-C₄ alkyl, —O (cyclopropyl), —O (allyl), allyl, —O(R₁), or —C(O) (R₁);
G₃ is unsubstituted C₁-C₃ alkyl or C₁-C₃ alkyl substituted by one, two, or three halogen atoms;
G₄ is unsubstituted C₁-C₃ alkyl or C₁-C₃ alkyl substituted by one, two, or three halogen atoms;
G₅ is selected from azetidine, pyrrolidine, morpholine, piperidine, or quinuclidine, each of which is optionally substituted by one or two substituents independently selected from methyl or halogen;
G₆ is selected from allyl; cyclopropyl; unsubstituted C₁-C₅alkyl; or C₁-C₅alkyl substituted either by one phenyl or by one, two, or three halogen atoms;
R₁ is unsubstituted C₁-C₄ alkyl or C₁-C₄ alkyl substituted by one, two, or three halogen atoms.

3. The process according to claim 2, wherein
G₁ is unsubstituted phenyl;
G₂ is unsubstituted phenyl;
G₃ is methyl;
G₄ is methyl;
G₅ is selected from azetidine, pyrrolidine, morpholine, piperidine, or quinuclidine, each of which is optionally substituted by one or two substituents independently selected from methyl or fluorine;
G₆ is selected from allyl, cyclopropyl, unsubstituted C₁-C₅alkyl, or C₁-C₅alkyl substituted either by one phenyl or by one, two, or three fluorine atoms; and
R₁ is unsubstituted C₁-C₄ alkyl or C₁-C₄ alkyl substituted by one, two, or three fluorine atoms.

4. The process according to claim 2, wherein the chiral amine (c) is the compound of formula (IV-a), 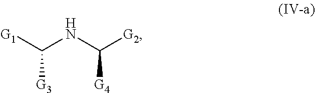
wherein:
G₁ and G₂ are each unsubstituted phenyl; and
G₃ and G₄ are each methyl.

5. The process according to claim 1, wherein the compound of formula (I-a) in the mixture is present in an enantiomeric excess over the compound of formula (I-b) of equal to or greater than 10%.

6. The process according to claim 1, wherein the compound of formula (I-a) in the mixture is present in an enantiomeric excess over the compound of formula (I-b) of equal to or greater than 80%.

7. The process according to claim 1, wherein the lithium base (d) is selected from Li—C₁-C₆-alkyl, Li—C₅-C₆-cycloalkyl, Li (aryl), LiH, LiNH₂, or lithium metal (Li⁰).

8. The process according to claim 1, wherein the lithium base (d) is selected from n-BuLi, sec-BuLi, i-PrLi or t-BuLi.

9. The process according to claim 1, wherein the aprotic solvent (f) is selected from THF, 1,4-dioxane, 2-methyltetrahydrofuran, diethyl ether, MTBE, or CPME.

10. The process according to claim 1, wherein the aprotic solvent (f) is THF.

11. The process according to claim 1, wherein the lithium salt (e) is present.

12. The process according to claim 11, wherein the lithium salt (e) is LiCl or LiBr.

13. The process according to claim 1, wherein X is cyclopropyl.

14. The process according to claim 1, wherein X is difluoromethyl.

15. The process according to claim 1, wherein X is trifluoromethyl.

16. The process according to claim 1, wherein Y is selected from —F, —Cl, —Br, —I, —CN, —O—C₁-C₆ alkyl, —O—C₃-C₆ cycloalkyl, —O(CO) C₁-C₆ alkyl, —O(CO) C₃-C₆ cycloalkyl, —O-aryl, or —O-heteroaryl; wherein-O(CO) C₁-C₆ alkyl or —O(CO) C₃-C₆ cycloalkyl may optionally be substituted by 1, 2, or 3 fluorine atoms, and wherein O-aryl and O-heteroaryl may be optionally substituted 1, 2, or 3 times by substituents independently selected from halogen atoms or C₁-C₃ alkyl.

17. The process according to claim 1, wherein Y is selected from —Cl, —OMe, or —OEt.

18. The process according to claim 1, wherein the process further comprises separating the compound of formula (I-a) from the mixture.

19. The process according to claim 18, wherein separating the compound of formula (I-a) from the mixture is achieved by trituration.

20. A compound of formula (I-a), 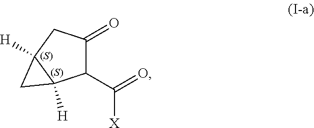
produced by the process according to claim 1, wherein X is as defined therein.
